(19) United States
(12) Patent Application Publication
(10) Pub. No.: US 2023/0030326 A1
(43) Pub. Date: Feb. 2, 2023

(54) SYNCHRONIZED BREEDING AND AGRONOMIC METHODS TO IMPROVE CROP PLANTS

(71) Applicant: PIONEER HI-BRED INTERNATIONAL, INC., JOHNSTON, IA (US)

(72) Inventors: Mark COOPER, St Lucia, IA (US); Carlos MESSINA, Gainesville, FL (US); Chunquan TANG, Ames, IA (US)

(73) Assignee: PIONEER HI-BRED INTERNATIONAL, INC., JOHNSTON, IA (US)

(21) Appl. No.: 17/658,351

(22) PCT Filed: Oct. 9, 2020
(86) PCT No.: PCT/US2020/054943

§ 371 Date: Apr. 7, 2022

Related U.S. Application Data
(60) Provisional application No. 62/913,686, filed on Oct. 10, 2019.

(51) Int. Cl. A01H 1/04; G16B 5/20; G16B 20/40; G16B 40/00

(52) U.S. Cl. CPC A01H 1/04; G16B 5/20; G16B 20/40; G16B 40/00

(57) ABSTRACT

Systems and methods that integrate breeding and agronomy by employing genotype (G) by environment (E) by management (M) practice to improve synchronized breeding for crop yield gain are provided. Methods to perform G×E×M through machine learning, simulation, crop models, quantitative models and other prediction techniques are provided.

### FIELD

The field relates to plant molecular genetics, breeding and agronomy for yield improvement.

### BACKGROUND

Agricultural production depends on a variety of factors—genetics, breeding populations, agronomy, and other factors that impact crop yield, including grain yield. Breeders create products, for example maize hybrids, but they are not actively selected to express the potential of a particular hybrid tailored to a desired agronomic practice or management technique. At the time when selection needs to be applied during breeding development, the desired agronomic practice is generally not known at a level that can make a greater impact. Agronomists develop such management practices for finished crop varieties (e.g., maize hybrids) that have already been developed by the breeder and whose genetic characteristics are relatively fixed compared to early-stage breeding population. There exists a need to improve crop yield by synchronized approaches to breeding in combination with agronomic practices at an earlier stage in the breeding process instead of a sequential approach dealing with late-stage finished commercial or pre-commercial genetic material.

### SUMMARY

Systems and methods to enable synchronized breeding and agronomic parameters improvement based on prospective analyses of current and future production systems and design of novel cropping systems based on outcomes from simulation and/or observations.

Systems and methods to identify genotype, management and genotype-by-management technologies to increase productivity of crops, cropping systems and agricultural systems for any set of target environmental conditions are disclosed.

Systems and methods to prioritize one or more parameters and experimental designs to breed for genotype, and genotype-by-management technologies for any crop that are specifically tailored to a target population's environmental conditions, geographical locations, and current stage of the breeding program and agronomic knowledge, such as for example, historical agronomic practice conditions.

Systems and methods for selection of individuals in a breeding pipeline tailored to pre-selected agronomic management parameters for improved performance that are targeted to one or more locations, conditions, and or management practices. For example, selection of plant populations occurs at an earlier stage (e.g., precommercial stage; or soon after early selections, one, two, three years after line coding). Selection can also be made at breeding development stage that is considered pre-coding (stage at which a line is designated having a commercial potential/value for further evaluation and/or development) occurs for individuals and/or populations. Selection may also occur at or before when a particular line is suitable as a breeding pair, e.g., crossing stage to generate populations for further breeding for genotype-by-management.

Systems and methods develop, produce, select, identify, characterize, screen genotypes where genotype refers to genetic components associated with one of multiple differences in haplotypes or DNA sequences for a given species or crop or among species or crops that encompass the cropping system or combination of cropping systems that encompass the agricultural system, which includes for example management practices.

Agronomic practices that are synchronized with an early-stage breeding program include such as for example: irrigation, planting date, plant population, planting density, plant nutrition, plant growth and/or development regulators, crop protection chemistry, biologicals, defoliation, harvest, crop sequence, crop rotations, crop combinations in one field, one farm, one geography or multiple fields, farms and geographies, or a combination of the foregoing.

Methods to combine agronomic characteristics to integrate, synchronize with breeding methods include e.g., methods based on crop growth models, statistical models including machine learning, remote sensing, and any combination suitable to generate a genotype×environment, genotype×management, and genotype×management systems.

Systems can be combined with optimization and breeding simulation to improve breeding-agronomy strategies to improve from a current productivity state to the desired productivity state defined by genotype and management. Methods to develop combination of genetic improvement and gap analyses to inform product creation, evaluation, commercialization for use at farmer fields can contribute to improve rates of genetic gain.

Systems and methods provided herein apply from plot to field to farm to multiple farms in one geography or multiple geographies across the globe. Systems and methods also apply to selection of a target population of genotype×management solutions defined as targets for genetic improvement and agronomy.

Systems disclosed herein can be combined with optimization and breeding simulation to define breeding-agronomy strategies in order to improve from a current productivity state to the desired productivity state defined by genotype and management. Systems and methods are provided to generate genotype×management solutions for consideration as targets for joint genetic and agronomic improvement.

Systems are provided to visualize target population of environments and systems, genetic gain, agronomic and genotype joint productivity improvement for prospective and retrospective analyses.

Systems and methods provided herein enable retrospective analyses of genetic gain and agronomic management can facilitate formulate breeding objectives for one crop such as improvement for drought tolerance and/or yield potential; for jointly formulate breeding objective such as breeding for one crop-management system for one target environment. Objective can be formulated as, for example—improve drought tolerance for rainfed sorghum when less than 200 mm of evapotranspiration is available, improve drought tolerance for limited irrigated maize when more than 200 mm of evapotranspiration but less than 400 mm is available, improve yield potential for maize when more than 400 mm but less than 800 mm is available, maturity of maize and soybean combined with defoliation treatment to fit a growing season when more than 800 mm of evapotranspiration is available in the system.

Similar to the evapotranspiration example, this is generalized to any nutrient or combination of nutrients such as nitrogen, phosphorous, potassium, sulfur and other micro nutrients.

Systems and methods provided herein enable prospective analyses and design of novel cropping systems based on outcomes of simulation and definition of joint breeding and agronomy objectives.

A specialized computing system for integrated breeding parameters and agronomic management practice, the system comprising: a memory; a first deep learning network stored in the memory, configured to compute first agronomy management practice effect on crop yield or genetic gain, the agronomy practice data as input;

a second deep learning network stored in the memory, configured to compute a second management practice effect on crop yield using the second management practice data as input;

a third deep network stored in the memory, configured to compute a third management practice effect on crop yield using the third management practice data as input;

a master deep learning network stored in the memory, configured to compute one or more yield values using the first, second, and third management practices effect on crop yield using the first, second, and third management practice data as inputs;

one or more processors communicatively coupled to the memory, configured to execute one or more instructions to cause performance of: receiving a particular dataset relating to one or more agricultural fields, wherein the particular dataset comprises particular first, second and third management practice data;

using the first deep learning network, computing the first management practice effect on crop yield for the one or more agricultural fields from the first management practice data;

using the second deep learning network, computing the second management practice effect on crop yield for the one or more agricultural fields from the second management practice data;

using the third deep learning network, computing the third management practice effect on crop yield for the one or more agricultural fields from the third management practice data; and

using the master deep learning network, computing one or more predicted yield values for the one or more agricultural fields from the first, second, and third management practice effects on crop yield.

In an embodiment, the first management practice data comprises nitrogen management; wherein the first deep learning network comprises a neural network configured to associations between the first management practice that are correlated to effects on crop yield. In an embodiment, the crop is maize, soy, canola, cotton, rice, wheat, sorghum, and sunflower. In an embodiment, the one or more breeding parameters include genotypic and/or phenotypic data. In an embodiment, the genotypic data includes a genome sequence information selected from the group consisting of SNP, QTL, RNA-seq, short read genomic sequencing, marker data, long read genome sequence information, methylation status, gene expression values, and indels.

In an embodiment, the agronomy management practice component is selected from the group consisting of irrigation, plant population density, planting date, nutrient application, seed or soil applied agricultural biologicals, crop rotations, and targeted in-season crop protection agent.

A method of identifying crosses for use in plant breeding, the method comprising:

accessing a dataset representative of multiple parents;

selecting, by a computing device, a subgroup of potential crosses, from the set of potential crosses, based on one or more thresholds associated with agronomy management scores for the set of potential crosses, each population prediction score associated with a predicted performance for a plurality of targeted agronomy management practices for the associated potential cross within the set of potential crosses;

selecting, by a computing device, multiple target crosses from the subgroup of potential crosses based on the performance of the parents in the targeted agronomy management practice environments;

ranking by a computing device, the target crosses based on a rule or an algorithm defining at least one threshold for a genotypic and/or phenotypic characteristics of one or more crosses; and

including a plant in a growing space of a breeding pipeline, the plant derived from at least one of the selected ones of the ranked target crosses.

In an embodiment, the agronomy management scores are based on one or more component selected from the group consisting of irrigation, plant population density, planting date, nutrient application, seed or soil applied agricultural biologicals, crop rotations, and targeted in-season crop protection agent.

### BRIEF DESCRIPTION OF THE DRAWINGS

FIG. 1 shows a schematic of sequential breeding and agronomy. In this approach, a small fraction of the agronomy-by-breeding space is explored. In this representation, this fact is indicated by the grey lines and arrows. Breeders and agronomists are generally not aware of the opportunities or the potential to increase yields through the combinations of management and breeding, especially when those techniques are synchronized and performed in a non-linear, non-sequential manner (e.g., represented by the white space within the box). Breeding research genotypes with higher performance are shown in dimension X for typical management defined by a state in dimension Yo.

FIG. 2 shows the synchronous breeding and agronomy approaches contemplated herein. In this approach, breeders and agronomists seek to characterize the agronomy-by-breeding space for opportunities to create genotype-by-management technologies. They seek to explore the white space and define the opportunities that become targets for creating genotypes combined with agronomy in one step. In this case, there may be multiple workable solutions attainable from any given starting point that they can seek to create. Dotted lines indicate feasible paths if sequential breeding-agronomy is pursue. None of the better solutions are accessible by following the path defined by the dotted lines.

FIG. 3 shows a representation of a simplified plant breeding cycle. Plants representing genotypes are sampled from the target population of genotypes for testing in field trials. Each trial will expose genotypes to a sample of environments possible drawn from the target population of environments. Phenotypes of interest are measured on the plants or crops in one or more trials. Analyses are conducted and based on the results the individuals are selected or discarded. The selected individuals are retained and used in a planned crossing scheme to create new progenies. When genotypic information is available, the breeder can use genomic prediction to predict values for traits of interest for all individuals for which he/she has seed available. In this way, he/she can increase the size of the breeding program. Agronomic management utilized to grow plants/crops is typical. Agronomists conduct trials where they change agronomic practices to provide recommendations for the growers that are tuned for the new genotype. This is a sequential process. When genotype-by-management interactions are significant, this sequential process can lead to reduced rate of genetic gain.

FIG. 4 shows synchronous breeding-agronomy technology development. This uses (as in sequential breeding) a process to sample genotypes from the target population of environments, grow plants in a sample of environments drawn from the target population of environments or generated in managed environments, analyse results, selects and continue the breeding cycle (1). However, this approach uses modelling and simulation to define the opportunities for genotype-by-management technologies in a target market or region (2). This simulation step informs breeding and agronomic objectives (3), thus the design of the field trials (4). Sets for prediction now include information for both genotype and management (5). With proper models (e.g., crop models) combined with genomic prediction prediction for genotypes available for the breeder could be assessed in context of different environments and management. As the cycle progresses, prediction is improved and more testing of genotype-by-management technologies is conducted rather than testing of samples of genotypes.

FIG. 5 shows another aspect of synchronous breeding-agronomy technology development. Steps towards creating yield clouds for defining breeding-agronomic objectives and assess created or predicted genotype-by-management technologies are shown in A (environment) and simulation/visualization combinations for genotype-by-management (B).

FIG. 6 shows approaches to defining breeding objectives and strategy-select for G or GxM. Use of mix models to determine variance components by environment/region (A) and predict opportunities to attain productivity goals as defined by quantiles 80 and 99 for yield at a given level or environmental resource based solely on genotype, management and genotype-by-management (B).

FIG. 7 shows representation of a simulation example (A) and results from the evaluation in the field (B) of genotypes and management. In the experimental case, the observations come from varying timing of irrigation. The two hybrids can be evaluated relative to the quantile fronts. Irrigation management could be optimized for each hybrid. In the case of prediction, these could become genotype-by-management options for field evaluation.

FIG. 8 shows breeding strategies based on opportunities to attain yields and how breeding contributed to increase yield within the genotype-by-management-by-environment space. (A) Analyses of experimental data (multiple experiments) to estimate rate of genetic gain. Colors represent different periods with unique characteristics related to breeding objectives, rate of genetic gain, and others. (B). Analyses of experimental data (each dot represents one hybrid in one experiment conducted under varying water regimes), within the yield-evapotranspiration framework. Each line is a quantile for a unique breeding period as shown in (A). (C) Project yield-evapotranspiration response curves for desired quantile for each breeding period. This projection can inform breeding objectives. For example, the largest genetic gain was attained under higher ET.

FIG. 9 shows about 35 environments created from the different combinations of plant population, irrigation quantity and timing, location and year sampled a diverse range of water availability regimes that differed in total ET and timing of water deficit as measured by the modelled Supply/Demand (S/D) ratio.

FIG. 10 shows about environments (42-59) created from the different combinations of plant population, irrigation quantity and timing, location and year sampled a diverse range of water availability regimes that differed in total ET and timing of water deficit as measured by the modelled Supply/Demand (S/D) ratio.

FIG. 11 shows (A) comparison between the experimental grain yield (GY) and season-long total evapotranspiration (ET) from planting to physiological maturity for the yield potential (open symbols) and flowering window (closed symbols) experiments and the predicted grain yield from the GY-ET 99% and 80% quantile regression negative exponential functions (equation 1) obtained from the large sample of genotype by environment by management (G×E×M) scenarios representing the US corn belt and (B) Modeled daily time-step water supply to demand (S/D) ratio and season-long total evapotranspiration (ET) from planting to physiological maturity for six environments (E36 to E41 Table 1) used to evaluate grain yield of two elite maize hybrids under a set of limited irrigation conditions managed to generate different levels of water deficit around flowering. E36_WW received the largest irrigation application and was used as a well-watered control relative to the sequence of five stress (S1 to S5) treatments. The five stress treatments are identified in sequence from S1 to S5 together with the target growing degree days window for imposition of the water deficit by withholding irrigation, e.g. E37_S1_400-1150 identifies environment 37 (E37), the first in the sequence of stress treatments (51) with irrigation withheld during the target window of 400 to 1150 growing degree days.

FIG. 12 shows spatial variation in genotype (Vg) management and genotype-by-management (Vgm) components.

FIG. 13 shows distribution of mean and standard deviation of simulated grain yield and ET across 2265 30 km×30 km grids used to represent the US corn belt together with boxplots of variance components from analyses of variance conducted for each of the 2265 grids.

FIG. 14 shows ratios of variance components for grain yield and ET for each of the 2265 30 km×30 km grids used to represent the US corn belt.

FIG. 15 shows scatter plots of G_BLUPs, M_BLUPs and G×M_BLUPs for grain yield and ET for (a.) grid 11349 selected based on largest VC ratio Vg/Vm, and (b.) grid 7453 selected based on largest VC ratio Vg×m/(Vg+Vm).

### DETAILED DESCRIPTION

The current disclosure provides systems and methods for increasing yield and/or improved agronomic performance based on improved breeding methods and agronomic practices.

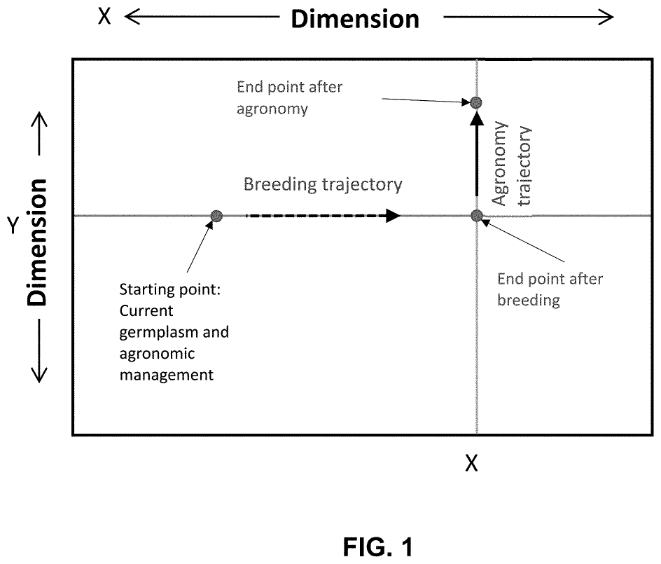

Advancement decisions in production agriculture seeking to improve crop productivity generally include two methodologies: (i) breeding increases yield potential and yield stability, and (ii) gap analyses diagnoses yield deviations and their frequencies from attainable yields to inform changes in agronomic management. These two methodologies are applied separately by breeders and agronomists in a sequential manner, but not in a systematic fashion where breeding and agronomic practices are integrated and synchronized at an earlier stage in the pipeline. If one considers breeding and agronomy as two separate disciplines or exploring technologies for superior performance in farmer's fields along sides of a square, then this sequential approach is equivalent to a walking towards a somewhat known destination without a map and following signs on the street while ignoring superior technologies that may reside out of the sidewalk (FIG. 1).

Irrigation, plant population density, planting date, nutrient application (e.g., N, P, K), other seed applied/soil applied components such as seed treatments, agricultural biologicals, crop rotations, and other practices form the agronomy management practice described herein.

In illustrated embodiments, water productivity and yield of maize (Zea mays L.) within the U.S. corn-belt were analyzed to develop solutions for integrated framework for predicting pathways to accelerate improvements in crop productivity through exploiting breeding and agronomy opportunities associated with G×E×M interactions.

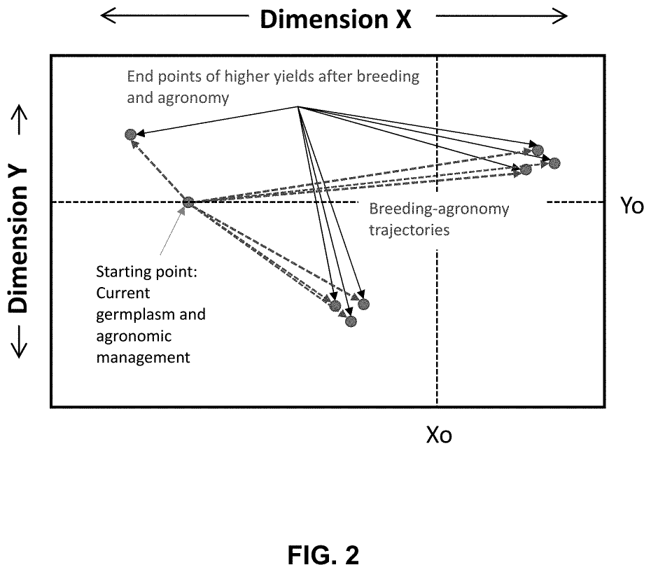

A more integrated framework that explores strategies for improvement of on-farm crop yield productivity from a Genotype by Environment by Management (G×E×M) perspective open new opportunities to design “end-to-end” crop improvement strategies that integrate the benefits of genetic gain (breeding) and gap analysis (agronomy) methodologies (FIG. 2). However, quantitative prediction frameworks that span both breeding and agronomy have not been demonstrated. The presence of G×E×M interactions both create opportunities for new prediction-based crop improvement strategies and provide certain requirements for the identification of desirable genotype-management combinations for the current dominant empirical research paradigm.

Opportunities to accelerate yield improvement may be overlooked because superior technologies (genotype and management) can reside outside the paths defined by classical or traditional breeding-agronomy sequential path (FIG. 2). By considering plausible technologies that reside in the “white” space, breeders and agronomists can create products linked to improved management for the set of environments that are relevant to the grower. These approaches provide options to develop improved products by management combinations that are superior to current options available to the grower that are generally limited to a breeding-then agronomy approach. Thus, methods and systems disclosed herein enable a non-sequential breeding-with-management practice versus a traditional breeding-then-management practice approach.

Systems and methods are provided herein to increase the benefits of integrating genetic improvement along with identifying suitable genotype-management combinations, in comparison to crop improvement processes that generally operate as an empirical sequential process where first the breeder identifies superior genotypes followed by a second step where the agronomist identifies superior management practices that can be applied in combination with the new genotypes.

Systems and methods for an integrated framework across breeding and agronomy to predict improvements in crop productivity from strategies that combine e.g., genetic gain, yield front and yield gap analysis are provided. In an embodiment, water productivity and yield of maize (Zea mays L.) within the US corn-belt was examined as a case study to develop the foundations for such an integrated framework for predicting pathways to accelerate improvements in crop productivity through exploiting breeding and agronomy opportunities associated with G×E×M interactions.

However, it is possible to analyse the results of genetic gain studies using the framework used for yield front and yield gap analysis. Advantages of this approach would include jointly considering: (1) the potential to increase productivity by breeding to improve yield potential across the whole yield front for a target population environment (TPE), (2) the potential to increase productivity by breeding to improve yield stability across the whole yield front for a TPE, (3) expanding opportunities to reduce the yield gap through identification of G, M, and GxM solutions and their combinations.

A biophysical framework is applied to investigate the design of crop improvement strategies with the potential for integrated contributions from breeding and agronomy. Water is a major resource that determines the productivity of all agricultural systems, including maize in the US corn-belt. Both breeding and agronomy can influence water use and the water productivity of agricultural systems. In certain cases, breeding and agronomy targets for a system are compared on a common basis, such as changes in water use required to achieve improvements in yield productivity. For example, comparing breeding strategies that change rates of canopy level transpiration and management strategies that change plant population could both be evaluated in terms of their impact on quantity and timing of water use from the soil profile and their independent and joint effects on crop yield. If this was done then it would be possible to investigate identification of desirable genotype-management combinations to achieve a target level of crop water productivity and water balance to realise the potential yield productivity of environments based on the crop available water, either through rain or irrigation. Further, it would then be possible to rank the different breeding and agronomy options for their feasibility, cost and short and long-term advantages as sustainable crop productivity improvement strategies.

In an embodiment, one option is to apply a maize crop growth model (CGM) to demonstrate a targeted simulation of grain yield G×E×M scenarios for the maize TPE of the US corn-belt. The simulation results are used to define the expected yield potential front and yield gap distributions associated with water productivity and the impact of water limitations. Another objective is to analyse three maize experimental studies for comparison with the CGM simulated G×E×M scenarios and their predicted yield potential front and yield gap distributions. The three experimental studies were (1) a maize ERA hybrid study to measure long-term genetic gain from breeding, (2) a maize yield potential study, and (3) a maize flowering drought stress study. The third objective is to use the results obtained from the simulation of G×E×M for grain yield of maize for the US corn-belt TPE and the comparisons with the experimental results to discuss opportunities for applying an integrated approach across breeding and agronomy to enhance understanding and prediction of G×E×M interactions and the creation and identification of desirable genotype-management combinations that improve maize yield productivity and stability by mitigating the negative effects of drought across the US corn-belt.

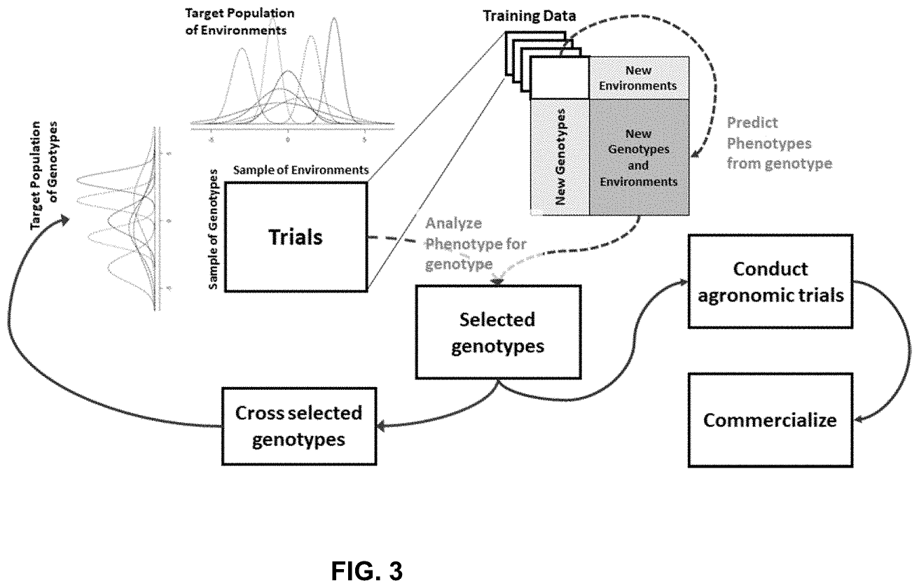

A simplified breeding program is considered. In such program, plants representing genotypes are sampled from the target population of genotypes for testing in field trials (FIG. 3). Each trial will expose genotypes to a sample of environments drawn from the target population of environments. Phenotypes are measured on the plants or crops in one or more trials to generate the data for analyses and evaluation against breeding objectives. Analyses are conducted and based on the results the individuals are selected or discarded. The selected individuals are retained and used in a crossing schemes designed by the breeder to create new progenies. When genotypic information is available, the breeder can use genomic prediction to predict values for traits of interest for a substantial portion or all of the individuals for which seed is available. In this way, the size of the breeding program is increased. Training sets are created specifically for prediction or created from trials conducted with other purposes. Agronomic management utilized to grow plants/crops is typical. Agronomists conduct trials where they change agronomic practices to provide recommendations for the growers that are tuned for the new genotype. The farmer further optimizes the agronomic management according to the characteristics of the farmer's operation. This is a sequential process. When genotype-by-management interactions are significant, this sequential process leads to reduced rate of yield gain.

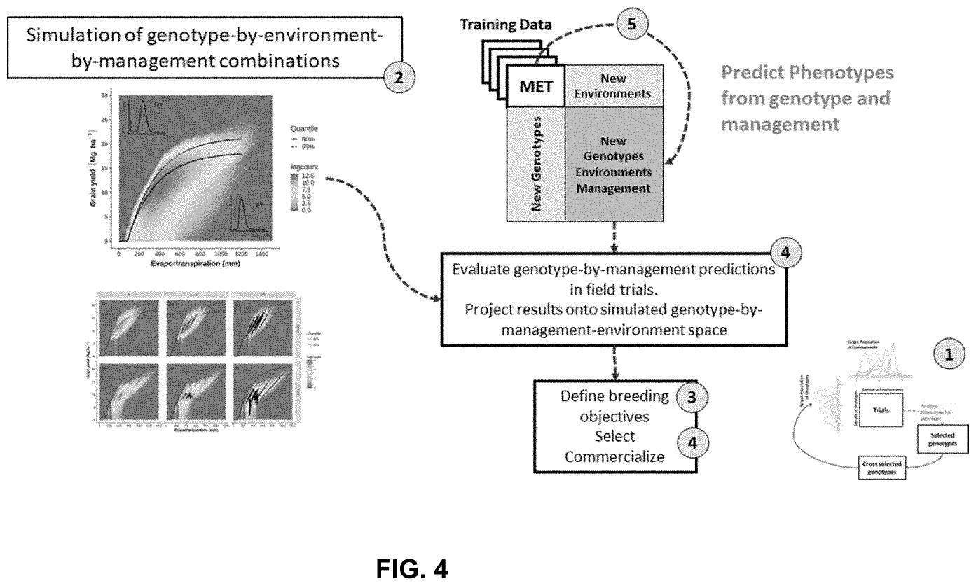

The proposed method, herein referred as “synchronous breeding and agronomy (SBA)”, uses a process to sample genotypes from the target population of environments, grow plants in a sample of environments drawn from the target population of environments or generated in managed environments, analyse results, select and continue the breeding cycle as described in FIGS. 3 and 4 (FIG. 4, (1)) and in a similar manner as sequential breeding does. In contrast to sequential breeding, the proposed method (SBA) uses modelling and simulation to define the opportunities for genotype-by-management technologies in a target market or region (FIG. 4, 2). This simulation step informs breeding and agronomic objectives and strategies (FIG. 4, 3). Therefore, the design of the field trials (FIG. 4, 4) is based on predictions and hypothesis about feasible technologies. Experimental sets for prediction now include information for both genotype and management (FIG. 4, 5) relevant to the target geographies. With proper models (e.g., crop models) combined with genomic prediction, prediction for genotypes available for the breeder could be assessed in context of different environments and management combinations. As the cycle progresses, prediction is improved, and more testing of genotype-by-management technologies is conducted to explore the “white” and technology space rather than testing of samples of genotypes.

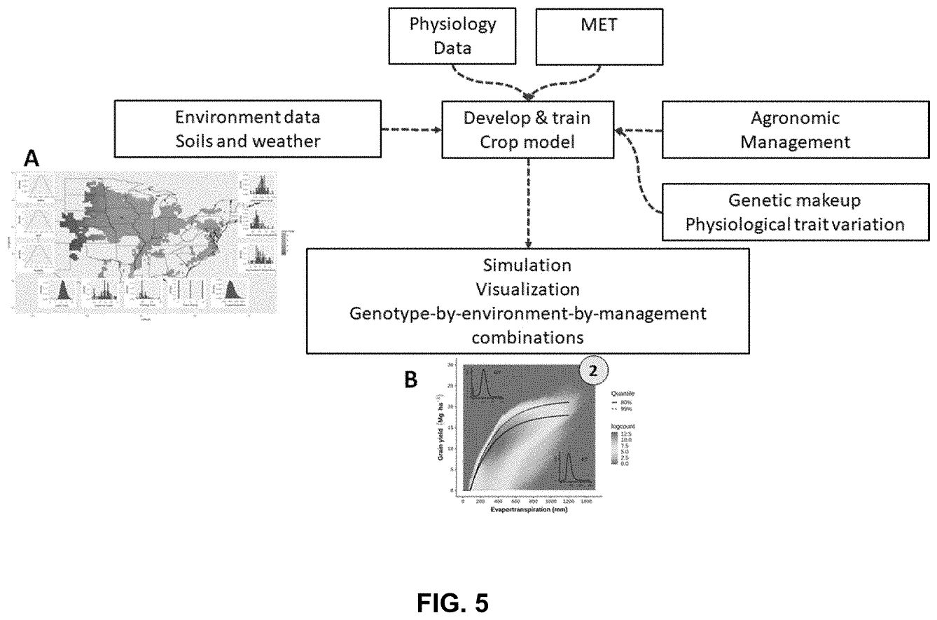

FIG. 5 illustrates the steps towards creating the genotype-by-management space that it is utilized to define the opportunities for genotype-by-management technologies in a target market or region, project results and evaluate merit of different genotype-by-management alternatives. Crop physiology experiments, data from multi-environment trials are used to develop a suitable crop models to predict performance of genotypes for the species, the variation of traits in the germplasm, environmental conditions and agronomic practices of interest. The outcomes of simulation could be visualized as a cloud representing the target population of genotypes, management, environment and interactions (FIG. 5, 2).

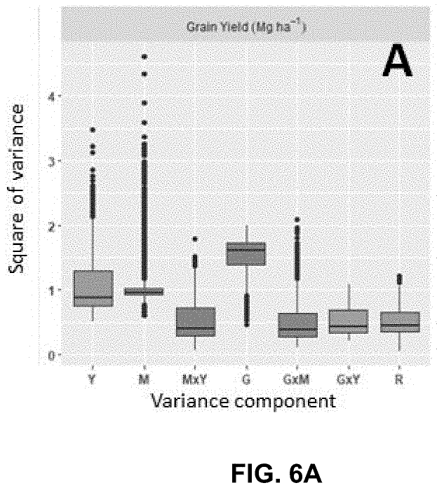

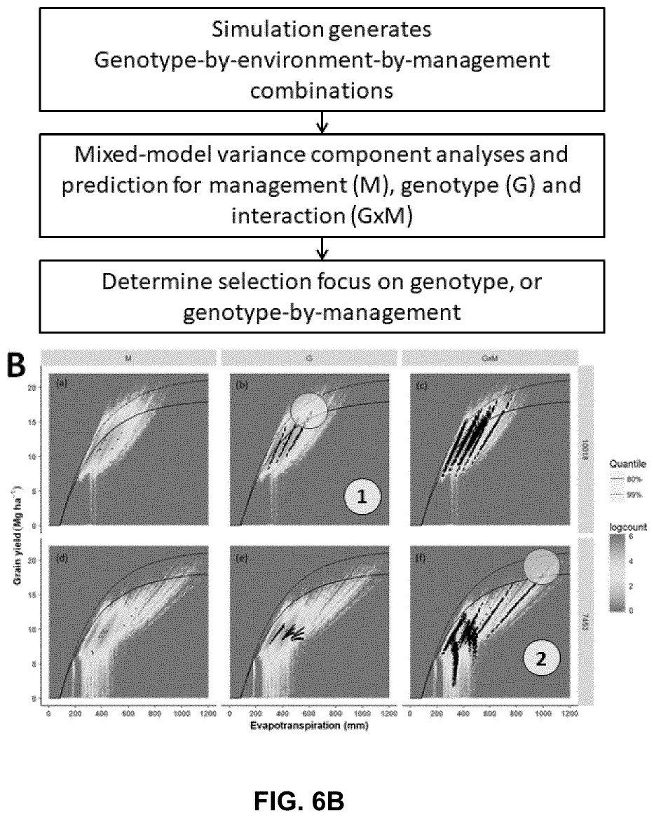

With the goal of defining breeding and agronomic objectives based on data and knowledge, outputs from simulation are analysed within a mixed model framework to estimate the contributions of genotype, management and genotype-by-management factors to the total variation (FIG. 6a). For a given grid/location, even production field, it is possible to make predictions for management, genotype and interaction. Then, a projection of this predictions (black dots in FIG. 6b) onto the space of possibilities, can help the breeder and agronomist assess which strategy is suitable for the geography/production system. Two cases are presented in the example. In case 1, the breeder can create genotypes that without major consideration for management can produce yields within yield quantiles 80 and 99—this is the target production space for high production efficiency and sustainable intensification (FIG. 6b). In case 2, breeding along or breeding followed by agronomic optimization cannot create genotypes to achieve this level of productivity in the absence of the correct agronomic management that is simulatenously practiced during the selection process of the breeding cycles (FIG. 6b). Only genotype-by-management combinations can produce yields at target efficiencies—for example yield between quantiles 80 and 99. Synchronous breeding-agronomy is most suitable to improve productivity gains in this type of production environment and cropping system. The method presented here enables the breeder and agronomist to make this first decision. Then take the predicted combinations and evaluate in the field the best combinations. The synchronous breeding-agronomy system is based on prediction and knowledge-based modelling.

“Synchronous Breeding-Agronomy method” includes for example: integration of gap analyses methodology and genetic gain methods. It uses modelling and prediction to create a set of opportunities to create superior products and solutions for the farmer. The method is demonstrated with 1) genetic gain studies conducted in maize with successful hybrids commercialized along a century of plant breeding, 2) hybrids with contrasting levels of drought tolerance grown in a range of water deficit conditions, and 3) biophysical simulation. Characterizing G×E×M interactions for a crop where a crop could be maize for a trait of interest, which could be but not restricted to yield for many G×E×M in any geography such as the US corn belt.
(i) A crop growth model, mechanistic or otherwise, capable of predicting effects of genetic, environmental and agronomic management manipulation generates outcomes to construct a map resulting from G×E×M interactions. The model generates yield or any metric of economic value or interest to the grower or decision maker and a metric of environmental variation or resource variation of interest to the decision maker. This could be evapotranspiration but not restricted to this metric. Databases feeds models with appropriate agronomic management, soils, genotypic information and other information to exercise the model (FIG. 5). The G×E×M space could be applied to multiple crops in which case the G term has a crop dimension and a genotypic dimension (e.g., hybrids for maize, varieties for soybean).
(ii) Defining the target genotype×management×environment space, attainable and potential repeatable yields, and variance components

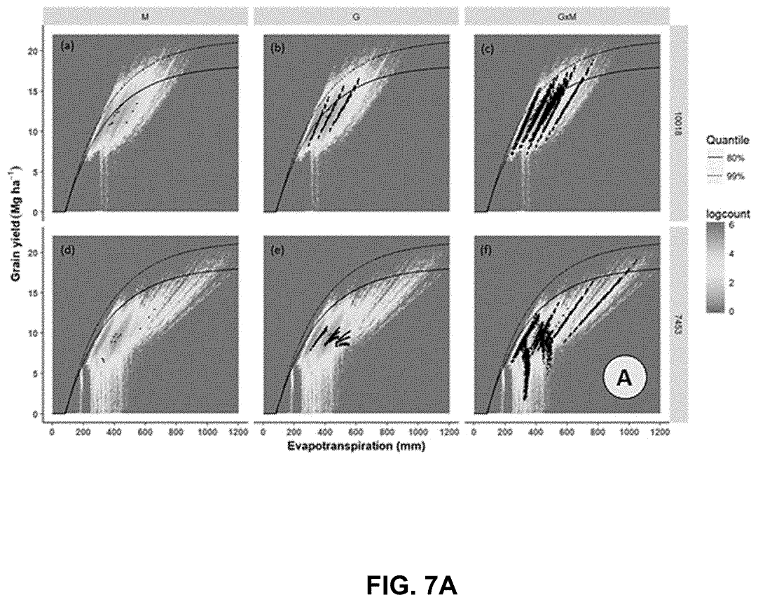

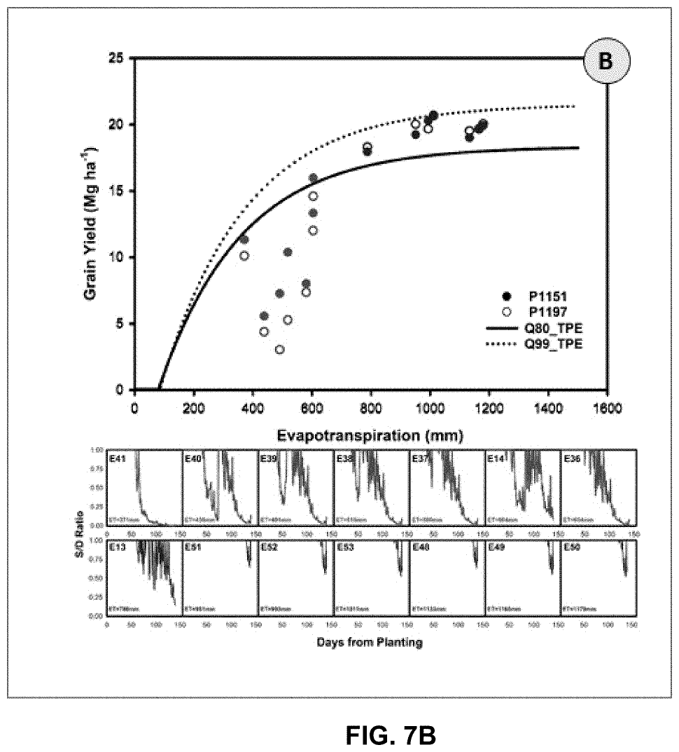

Using outputs from modelling and simulation listed in step 1, one applies gap analyse methodology, namely 1) determination of fronts calculated using quantile regression (FIG. 5b), and 2) projection of any empirical data if available to interpret both experimental observations, simulations and empirical G×E×M analyses conducted at the same data (FIG. 6b; FIG. 7b), and 3) estimate variance components for each grid or geographical unit of interest (FIG. 6a).

Simulations are represented for example as a heat map depicting the target population of environments, or more generally, the set of environments that are of interest to the decision maker. Quantile regression is utilized to define boundaries, 99, 90, 80 percentiles which are common boundaries utilized in gap analyses (FIG. 5b, dotted and solid lines). Other boundaries of interest could be defined. These boundaries define the regions of successful crop performance. In the method presented the regions are extended to outcomes of agricultural or cropping system or crop performance (FIG. 5).

Analysis of sources of variance for each grid and summarisation of the results for the full set of grids. At the grid level, a mixed model analysis of the simulated grain yield and evapotranspiration data (ET) data was conducted applying the model (with all terms except for mu treated as random):

Tᵢⱼₖ=mu+Gᵢ+Mⱼ+Yₖ+(GM)ᵢⱼ+(GY)ᵢₖ+(MY)ⱼₖ+eᵢⱼₖ

where Tᵢⱼₖ is the Trait (Grain yield or ET) value for genotype i in management j in year k, mu is the fixed effect for the overall mean, Gᵢ is the main-effect for genotype i, assumed to be N(0,σ2G), Mⱼ is the main-effect for management j, assumed to be N(0, σ2M), Yₖ is the main-effect for year k, assumed to be N(0, σ2Y), (GM)ᵢⱼ is the Genotype-by-Management interaction effect for Genotype I and Management j, assumed to be N(0, σ2GM), (GY)ᵢₖ is the genotype-by-year interaction effect for Genotype I and Year k, assumed to be N(0, σ2GY), (MY)ⱼₖ is the Management-by-Year interaction effect for Management j and Year k, assumed to be N(0, σ2MY), and eᵢⱼₖ is the residual effect for Genotype I in Management j and Year k, assumed to be N(0,σ2e).

These variance components provide the first views and assessments for the opportunities to close the gap using genotype-management technologies. Boxplots could help visualize how variance components change with geography or any other metric of interest (FIG. 6a).

Definition of target population of genotype×management solutions by projecting empirical datasets onto digital maps. Empirical datasets for a crop or cropping systems or agricultural systems are utilized to assess the boundaries of the theoretical space and to evaluate the relative merits of alternative Genotype, Management and Genotype-Management technology options to achieve target levels of on-farm crop productivity. These empirical datasets are generated, but not restricted to experimentation under controlled conditions with the purposes of 1) developing models, 2) test predictions for genotype-by-management technologies, 3) evaluate genotypes, and 4) the construction of training sets, among other purposes. Farmers data could be projected onto heat maps to evaluate simulations and diagnose gaps and frequencies (FIG. 7). Characterization of temporal dynamics of water deficit can help diagnose and identify genotype-by-environment-by-management opportunities for improved productivity (FIG. 7b).

Projection of empirical datasets help breeders and agronomist define the actual space and opportunities for joint genetic-agronomic improvement. The comparison between these actual points extracted from the real world and the simulated genotype-by-management virtual points, grids or otherwise, provide clear targets for improvement. Breeding simulation, optimization algorithms, or simple heuristic approaches could be used to define the path from actual to future states.

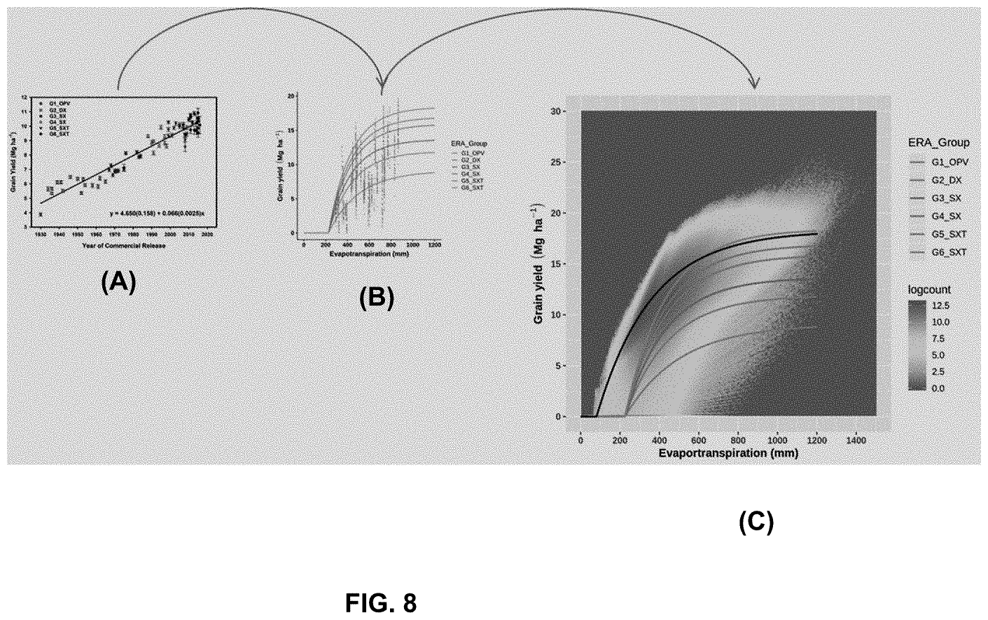

FIG. 8 shows how to utilize analyses of experimental data generated to monitor genetic gain to inform decisions. First, the data is projected onto the yield-evapotranspiration space, the x-axis could be any resource of interest to the farmer, agronomy or the breeder, and it could be multivariate as well as the y-axis. Once genetic gain is established in the gap analyses framework, results are overlay within the theoretical space. Analyses are conducted to evaluate opportunities to continue improving yield potential (hi values of ET), intermediate and hi levels of drought stress. The farmer, breeder and agronomist can now define strategies to determine future paths for genetic improvement. If for a given crop there was limited genetic gain under drought, the strategy could consist in focusing breeding efforts in other crops. If genetic gain for very high ET is limited, stakeholders can seek breeding strategies to enable new cropping systems to leverage multi-crops.

Agronomic management practice includes modeling various agronomic parameters such as different types of inputs, including crop type, soil type, weather, environmental classifications, and other management practices, that can influence crop yield. Some of these inputs like temperature vary temporally, while other inputs, like soil type, vary spatially.

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein, the term “allele” refers to a variant or an alternative sequence form at a genetic locus. In diploids, single alleles are inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the phrase “associated with” refers to a recognizable and/or assayable relationship between two entities. For example, the phrase “associated with a trait” refers to a locus, gene, allele, marker, phenotype, etc., or the expression thereof, the presence or absence of which can influence an extent, degree, and/or rate at which the trait is expressed in an individual or a plurality of individuals.

As used herein, the term “backcross”, and grammatical variants thereof, refers to a process in which a breeder crosses a progeny individual back to one of its parents: for example, a first generation F₁ with one of the parental genotypes of the F₁ individual.

As used herein, the phrase “breeding population” refers to a collection of individuals from which potential breeding individuals and pairs are selected. A breeding population can be a segregating population.

A “candidate set” is a set of individuals that are genotyped at marker loci used for genomic prediction. The candidates may be hybrids.

As used herein, the term “chromosome” is used in its art-recognized meaning as a self-replicating genetic structure containing genomic DNA and bearing in its nucleotide sequence a linear array of genes.

As used herein, the terms “cultivar” and “variety” refer to a group of similar plants that by structural and/or genetic features and/or performance can be distinguished from other members of the same species.

As used herein, the phrase “determining the genotype” or “analyzing genotypic variation” or “genotypic analysis” of an individual refers to determining at least a portion of the genetic makeup of an individual and particularly can refer to determining genetic variability in an individual that can be used as an indicator or predictor of a corresponding phenotype. Determining a genotype can comprise determining one or more haplotypes or determining one or more polymorphisms exhibiting linkage disequilibrium to at least one polymorphism or haplotype having genotypic value. Determining the genotype of an individual can also comprise identifying at least one polymorphism of at least one gene and/or at one locus; identifying at least one haplotype of at least one gene and/or at least one locus; or identifying at least one polymorphism unique to at least one haplotype of at least one gene and/or at least one locus. Genotypic variations may also include inserted transgenes or other changes engineered in the host genome.

A “doubled haploid plant” is a plant that is developed by the doubling of a haploid set of chromosomes. A doubled haploid plant is homozygous.

As used herein, the phrase “elite line” refers to any line that is substantially homozygous and has resulted from breeding and selection for superior agronomic performance.

As used herein, the term “gene” refers to a hereditary unit including a sequence of DNA that occupies a specific location on a chromosome and that contains genetic instructions for a particular characteristic or trait in an organism.

As used herein, the phrase “genetic gain” refers to an amount of an increase in performance that is achieved through artificial genetic improvement programs. The term “genetic gain” can refer to an increase in performance that is achieved after one generation has passed.

As used herein, the phrase “genetic map” refers to an ordered listing of loci usually related to the relative positions of the loci on a particular chromosome.

As used herein, the phrase “genetic marker” refers to a nucleic acid sequence (e.g., a polymorphic nucleic acid sequence) that has been identified as being associated with a trait, locus, and/or allele of interest and that is indicative of and/or that can be employed to ascertain the presence or absence of the trait, locus, and/or allele of interest in a cell or organism. Examples of genetic markers include, but are not limited to genes, DNA or RNA-derived sequences (e.g., chromosomal subsequences that are specific for particular sites on a given chromosome), promoters, any untranslated regions of a gene, microRNAs, short inhibitory RNAs (siRNAs; also called small inhibitory RNAs), quantitative trait loci (QTLs), transgenes, mRNAs, double-stranded RNAs, transcriptional profiles, and methylation patterns.

As used herein, the term “genotype” refers to the genetic makeup of an organism. Expression of a genotype can give rise to an organism's phenotype (i.e., an organism's observable traits). A subject's genotype, when compared to a reference genotype or the genotype of one or more other subjects, can provide valuable information related to current or predictive phenotypes. The term “genotype” thus refers to the genetic component of a phenotype of interest, a plurality of phenotypes of interest, and/or an entire cell or organism.

As used herein, “haplotype” refers to the collective characteristic or characteristics of a number of closely linked loci within a particular gene or group of genes, which can be inherited as a unit. For example, in some embodiments, a haplotype can comprise a group of closely related polymorphisms (e.g., single nucleotide polymorphisms; SNPs). A haplotype can also be a characterization of a plurality of loci on a single chromosome (or a region thereof) of a pair of homologous chromosomes, wherein the characterization is indicative of what loci and/or alleles are present on the single chromosome (or the region thereof).

As used herein, the term “heterozygous” refers to a genetic condition that exists in a cell or an organism when different alleles reside at corresponding loci on homologous chromosomes.

As used herein, the term “homozygous” refers to a genetic condition existing when identical alleles reside at corresponding loci on homologous chromosomes. It is noted that both of these terms can refer to single nucleotide positions, multiple nucleotide positions (whether contiguous or not), and/or entire loci on homologous chromosomes.

As used herein, the term “hybrid”, when used in the context of a plant, refers to a seed and the plant the seed develops into that results from crossing at least two genetically different plant parents.

As used herein, the term “inbred” refers to a substantially or completely homozygous individual or line. It is noted that the term can refer to individuals or lines that are substantially or completely homozygous throughout their entire genomes or that are substantially or completely homozygous with respect to subsequences of their genomes that are of particular interest.

As used herein, the term “introgress”, and grammatical variants thereof (including, but not limited to “introgression”, “introgressed”, and “introgressing”), refer to both natural and artificial processes whereby one or more genomic regions of one individual are moved into the genome of another individual to create germplasm that has a new combination of genetic loci, haplotypes, and/or alleles. Methods for introgressing a trait of interest can include, but are not limited to, breeding an individual that has the trait of interest to an individual that does not and backcrossing an individual that has the trait of interest to a recurrent parent.

As used herein, “linkage disequilibrium” (LD) refers to a derived statistical measure of the strength of the association or co-occurrence of two distinct genetic markers. Various statistical methods can be used to summarize LD between two markers but in practice only two, termed D′ and r², are widely used (see e.g., Devlin & Risch 1995; Jorde, 2000). As such, the phrase “linkage disequilibrium” refers to a change from the expected relative frequency of gamete types in a population of many individuals in a single generation such that two or more loci act as genetically linked loci.

As used herein, the phrase “linkage group” refers to all of the genes or genetic traits that are located on the same chromosome. Within a linkage group, those loci that are sufficiently close together physically can exhibit linkage in genetic crosses. Since the probability of a crossover occurring between two loci increases with the physical distance between the two loci on a chromosome, loci for which the locations are far removed from each other within a linkage group might not exhibit any detectable linkage in direct genetic tests. The term “linkage group” is mostly used to refer to genetic loci that exhibit linked behavior in genetic systems where chromosomal assignments have not yet been made. Thus, in the present context, the term “linkage group” is synonymous with the physical entity of a chromosome, although one of ordinary skill in the art will understand that a linkage group can also be defined as corresponding to a region (i.e., less than the entirety) of a given chromosome.

As used herein, the term “locus” refers to a position on a chromosome of a species, and can encompass a single nucleotide, several nucleotides, or more than several nucleotides in a particular genomic region.

As used herein, the terms “marker” and “molecular marker” are used interchangeably to refer to an identifiable position on a chromosome the inheritance of which can be monitored and/or a reagent that is used in methods for visualizing differences in nucleic acid sequences present at such identifiable positions on chromosomes. A marker can comprise a known or detectable nucleic acid sequence. Examples of markers include, but are not limited to genetic markers, protein composition, peptide levels, protein levels, oil composition, oil levels, carbohydrate composition, carbohydrate levels, fatty acid composition, fatty acid levels, amino acid composition, amino acid levels, biopolymers, starch composition, starch levels, fermentable starch, fermentation yield, fermentation efficiency, energy yield, secondary compounds, metabolites, morphological characteristics, and agronomic characteristics.

The term “phenotype” refers to any observable property of an organism, produced by the interaction of the genotype of the organism and the environment. A phenotype can encompass variable expressivity and penetrance of the phenotype. Exemplary phenotypes include but are not limited to a visible phenotype, a physiological phenotype, a susceptibility phenotype, a cellular phenotype, a molecular phenotype, and combinations thereof.

As used herein, the term “population” refers to a genetically heterogeneous collection of plants that in some embodiments share a common genetic derivation.

As used herein, the term “progeny” refers to any plant that results from a natural or assisted breeding of one or more plants. For example, progeny plants can be generated by crossing two plants (including, but not limited to crossing two unrelated plants, backcrossing a plant to a parental plant, intercrossing two plants, etc.), but can also be generated by selfing a plant, creating an inbred (e.g., a double haploid), or other techniques that would be known to one of ordinary skill in the art. As such, a “progeny plant” can be any plant resulting as progeny from a vegetative or sexual reproduction from one or more parent plants or descendants thereof. For instance, a progeny plant can be obtained by cloning or selfing of a parent plant or by crossing two parental plants and include selfings as well as the F₁ or F₂ or still further generations. An F₁ is a first-generation progeny produced from parents at least one of which is used for the first time as donor of a trait, while progeny of second generation (F₂) or subsequent generations (F₃, F₄, and the like) are in some embodiments specimens produced from selfings (including, but not limited to double haploidization), intercrosses, backcrosses, or other crosses of F₁ individuals, F₂ individuals, and the like. An F₁ can thus be (and in some embodiments, is) a hybrid resulting from a cross between two true breeding parents (i.e., parents that are true-breeding are each homozygous for a trait of interest or an allele thereof, and in some embodiments, are inbred), while an F₂ can be (and in some embodiments, is) a progeny resulting from self-pollination of the F₁ hybrids.

As used herein, the phrase “single nucleotide polymorphism”, or “SNP”, refers to a polymorphism that constitutes a single base pair difference between two nucleotide sequences. As used herein, the term “SNP” also refers to differences between two nucleotide sequences that result from simple alterations of one sequence in view of the other that occurs at a single site in the sequence. For example, the term “SNP” is intended to refer not just to sequences that differ in a single nucleotide as a result of a nucleic acid substitution in one as compared to the other, but is also intended to refer to sequences that differ in 1, 2, 3, or more nucleotides as a result of a deletion of 1, 2, 3, or more nucleotides at a single site in one of the sequences as compared to the other. It would be understood that in the case of two sequences that differ from each other only by virtue of a deletion of 1, 2, 3, or more nucleotides at a single site in one of the sequences as compared to the other, this same scenario can be considered an addition of 1, 2, 3, or more nucleotides at a single site in one of the sequences as compared to the other, depending on which of the two sequences is considered the reference sequence. Single site insertions and/or deletions are thus also considered to be encompassed by the term “SNP”.

As used herein, the terms “trait” and “trait of interest” refer to a phenotype of interest, a gene that contributes to a phenotype of interest, as well as a nucleic acid sequence associated with a gene that contributes to a phenotype of interest. Any trait that would be desirable to screen for or against in subsequent generations can be a trait of interest. Exemplary, non-limiting traits of interest include yield, disease resistance, agronomic traits, abiotic traits, kernel composition (including, but not limited to protein, oil, and/or starch composition), insect resistance, fertility, silage, and morphological traits. In some embodiments, two or more traits of interest are screened for and/or against (either individually or collectively) in progeny individuals.

Various methods can be used to introduce a genetic modification at a genomic locus that encodes and polypeptide into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

In addition to modification by a double strand break technology, modification of one or more bases without such double strand break are achieved using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

These fusions contain dCas9 or Cas9 nickase and a suitable deaminase, and they can convert e.g., cytosine to uracil without inducing double-strand break of the target DNA. Uracil is then converted to thymine through DNA replication or repair. Improved base editors that have targeting flexibility and specificity are used to edit endogenous locus to create target variations and improve grain yield. Similarly, adenine base editors enable adenine to inosine change, which is then converted to guanine through repair or replication. Thus, targeted base changes i.e., C⋅G to T⋅A conversion and A⋅T to G⋅C conversion at one more locations made using appropriate site-specific base editors.

In an embodiment, base editing is a genome editing method that enables direct conversion of one base pair to another at a target genomic locus without requiring double-stranded DNA breaks (DSBs), homology-directed repair (HDR) processes, or external donor DNA templates. In an embodiment, base editors include (i) a catalytically impaired CRISPR-Cas9 mutant that are mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; and (iv) nickase activity to cleave the non-edited DNA strand, followed by cellular DNA repair processes to replace the G-containing DNA strand.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

### EXAMPLES

The present disclosure is further illustrated in the following Examples. It should be understood that these Examples, while indicating embodiments of the invention, are given by way of illustration only. Thus, various modifications to the crop model, the relationships to simulate/model the limited transpiration trait, methods of analyses, and applying such methods for crop improvement are disclosed.

### Example 1

### Genotype-Environment-Management and Gap Analysis Methods Including Crop Modeling

A crop growth model (CGM) was used to conduct a simulation experiment. Other models could be used for this purpose. The objective of the simulation experiment was to sample and characterize G×E×M interactions for grain yield and canopy level evapotranspiration (ET) of maize hybrids within the context of the Target Population of Environments (TPE). The focus of the simulation experiment was on yield productivity for G×E×M combinations that sampled a range of water balance scenarios. Grain yield and ET were modelled for a sample of G×E×M scenarios used to represent maize crop yield productivity. The CGM was based on the mechanistic model for the demonstration of an example but it is not restricted to such model. Models are available for many crops (e.g., DSSAT, APSIM) and can be formulated in different ways including empirical relations.

In the present example, grain yield was simulated from the daily increase in harvest index ending at physiological maturity with mass simulated along the growth cycle using concepts of radiation and water use, and radiation and water use efficiencies. Soil properties, irrigation, precipitation, temperature, and solar radiation are environmental variables that are input to the model. The evapotranspiration (ET) was calculated by adding the evaporation component as described by Sinclair and the transpiration component that is calculated based on growth limited by solar radiation or water. Other approaches could be utilized to estimate ET. G×E×M scenarios were developed as follows (non-limiting examples):

The environmental (E) dimension of the US corn-belt TPE was described as a combination of geographical (location) and temporal (year) dimensions. The geographical dimension was defined by a set of 30×30 km grids (total of 2265 grids) used within an environmental classification system to define the row cropping areas of the US. A grid was identified as a 30×30 km grid that contained more than 3000 corn acres based on USDA data. Soil and weather variables were then defined for each grid to be used as inputs for the CGM. For each grid the dominant soil type, yearly initial soil water contents and yearly planting dates were extracted from databases. Daily weather data (maximum, minimum temperature and precipitation) from multiple sources (NOAA, HPRCC and research station network) was inverse distance interpolated for the centroid of each grid used in the simulation.

The management (M) dimension was described using a combination of irrigation strategies and plant populations. Four irrigation schemes and three plant populations were varied for the CGM simulations. The irrigation schemes were: (1) no irrigation; rainfed, (2) V12 irrigation; 20 mm minus precipitation for five days following the developmental stage of V12, (3) Weekly irrigation; irrigate to replace ET loss from the previous 5 days in two consecutive days, minus precipitation, maximum of 40 mm irrigation applied over two days, (4) Optimal irrigation; replace all ET losses each day. The plant population densities used for the CGM simulations were 6, 8 and 10 plants m-2. The 12 irrigation-density combinations were implemented for each of the 2265 30×30 km grids for each year.

The genetic (G) dimension for the current study was described by the factorial combination of a set of five traits selected based on empirical evidence demonstrating a contribution of genetic variation for the traits to genetic variation or grain yield among maize hybrids in water limited and favorable environments relevant to the US corn-belt. The five chosen traits were; (1) area of the largest leaf in the profile (AMAX), (2) Mass of the ear at silking (MEB), (3) Total leaf number (TLNO), (4) total solar radiation intercepted use (RueMax) and (5) restricted transpiration modeled as the slope of the vapor pressure deficit curve (vpd.slope; maximum value is one and the slopes are relative). To simulate genetic diversity for the traits five genetic parameters in the CGM were selected to express variation across three levels (Table 1). For the five traits and three levels for each trait there were 35=246 combinations of the trait input levels for the CGM. For each of the 246 trait combinations two maturity classes were identified, fixed and stratified. The fixed class was one maturity level held constant across all 2265 grids of the US corn-belt. The stratified class adjusted maturity level with latitude of the grid so that longer season maturity was used for the more southern latitudes and shorter season maturity was used for the more northern latitudes. Maturity is determined by the number of leaves, the rate of leaf appearance and the duration of the grain filling period. For each grid, the environmental classification system provides the typical maturity. Based on this information, for each grid, the parameters controlling grain fill, initial leaf number and leaf appearance rates were determined based on maturity group. These parameters were estimated for precommercial and commercial hybrids. Thus, 2×35=486 genotypes were generated from the combinations of the five traits and two maturity types. In addition to the 486 genotypes created by the trait combinations and maturity classes a CGM parameterization for the check hybrid P1151 was included and as for the other 486 genotypes two maturity classes were generated. Thus, a total of 488 genotypes were modeled to generate the genotypic (G) space studied.

TABLE 1
Maize crop growth model to sample the genotype
dimension for the simulation of grain yield.

| Trait Parameter | Value 1 | Value 2 | Value 3 |
|---|---|---|---|
| AMAX (cm²) | 700 | 900 | 1100 |
| MEB (g) | 0.6 | 0.9 | 1.2 |
| TLN (leaf number count) | 18 | 19 | 20 |
| RueMax (g/MJ⁻¹) | 1.60 | 1.85 | 2.10 |
| Vpd.slope (unitless) | 0.4 | 0.7 | 1.0 |

Three levels for the five traits used in combination with the maize Crop Growth Model to sample the Genotype dimension for the simulation of Grain Yield and Evapotranspiration for the GxExM scenarios within the maize Target Population of Environments representing the US corn-belt. AMAX determines the maximum potential area of the largest leaf in the canopy; larger values are associated with larger canopy size. MEB determines the ear biomass that is required before silks can emerge from the husks surrounding the ear; smaller values are associated with educed anthesis to silking interval and greater reproductive resiliency. TLN determines the total number of leaves on a plant; larger values are associated with larger canopy size. RueMax determines the efficiency with which canopy intercepted radiation is converted into biomass; larger values are associated with greater radiation use efficiency. Vpd.slope determines the canopy transpiration responsiveness to atmospheric Vapor Pressure Deficit (VPD); the slopes are relative with a value of 1.0 associated with no restriction on transpiration and lower values associated with restricted transpiration.

To simulate the G×E×M space for the US corn belt each of the 488 genotypes as tested for each of the 12 irrigation-density combinations for each of the 2265 grids for each year. The CGM was used to simulate both grain yield and evapotranspiration (ET) for each 30×30 km grid for each year for each management strategy and each genotype, resulting in approximately 663 million simulations. An example of the outputs from running the full set of simulations for one 30×30 km grid (latitude=41.684, longitude=−93.508) was chosen to illustrate the CGM outputs (FIG. 5a). The cumulative results for all 2265 30×30 km grids collectively define a dense sampling of potential G×M×E interactions for grain yield and ET for maize within the US corn-belt TPE. The simulated yield and ET results were subjected to additional analyses.

A heat map graphical representation of yield-ET associations for the TPE was constructed from the modeled G×E×M scenarios. The simulated yield-ET pairs were converted from points to a categorized heat map visualization. To create the heat map the yield data were sorted into 0.1 Mg ha⁻¹ categorical steps starting from 0 Mg ha⁻¹ up to the final category that included the highest yield data points. Similarly, the ET data were sorted into 5 mm steps starting from 0 mm up to the final category that included the highest ET data points. Whenever a yield data point or an ET data point coincided with the boundary point between two categories the data point was moved up into the higher yield and/or ET category. The number of data points within each yield-ET category was counted and the distribution of the counts across all segments was visualized on a color scale and the color intensity for the category was plotted to create the yield-ET heat map for the modeled G×E×M interactions of the TPE.

Quantile regression was used to estimate a yield potential front conditional on ET for the complete set of modeled Yield-ET for the G×E×M scenarios. Following exploratory comparisons between alternative functions a truncated negative exponential function was selected to fit the yield fronts. The function was constrained to zero if ET was less than ET₀. Therefore, the selected negative exponential function was

$${y}_{ET} = \{ \begin{matrix}0 , & if ET < {ET}_{\text{?}} \\ {Y}_{\text{?}} ( 1 - {e}^{- \text{?}} ) , & if ET \geq {ET}_{\text{?}}\end{matrix}$$

$$\text{?} \text{indicates text missing or illegible when filed}$$

where y_{ET} is the predicted yield for a defined level of evapotranspiration (ET), Yp is the yield potential, TE is the transpiration efficiency and ET₀ is the evapotranspiration at which yield is zero. The coefficients for the quantile regression functions were estimated using the interior point method as implemented in the function nlrq in the R package quantreg.

Following comparisons of different target quantiles, ranging from 80% to 99%, the truncated negative exponential function was estimated for the 80% and 99% quantiles. To accommodate the large size of the complete G×E×M data set for the TPE a bootstrap sampling strategy was applied. Following preliminary investigations, 12 bootstraps of 5% of the complete set of G×E×M scenarios were used to obtain an estimate of the 99% and 80% quantile regression of the yield potential front using the truncated negative exponential function. The coefficients of the quantile regressions for the complete TPE data set were estimated from the average of the 12 bootstraps and their standard error from the standard error of the 12 bootstraps. The estimates of the yield potential fronts obtained from the 80% and 99% quantile regressions were superimposed on the yield-ET TPE heat map to investigate the practical yield potential for maize within the US corn belt. The 99% quantile regression curve was used to provide predictions of potential yields for an environment based on the crop available water. The 80% quantile regression curve was used to provide predictions of the exploitable yield target based on crop available water. Therefore, hybrid yield outcomes for a given crop available water, as determined by ET, that are between the predicted grain yield levels for the 80% and 99% quantile regression functions are considered to be successful G×E×M outcomes. In contrast hybrid yield outcomes below the predictions of the 80% quantile regression predictions are considered to be unsuccessful G×E×M outcomes for gap analysis investigation with the objective of identifying alternative GxM combinations that could be adopted to improve the hybrid yield outcomes to higher levels between the levels predicted by the 80% and 99% quantile regressions for a given level of water availability.

A joint association between the modeled grain yield and evapotranspiration for the large sample of G×E×M scenarios was used to create the GY-ET heat map and independent frequency density distributions for grain yield and ET (FIG. 5b). The G×E×M scenarios generated a wide distribution of GY and ET values (FIG. 5b). The GY-ET heat map and the density plots for both GY and ET highlighted the high frequency of G×E×M scenarios resulting in ET levels between 200 and 700 mm resulting in GY outcomes between 7 and 17 Mg ha⁻¹. In general, there was a positive association between ET and GY over the full range of G×E×M scenarios. However, there were non-linear features of the association highlighted by the GY-ET heat map. The modelled GY ranged from 0 Mg ha⁻¹ up to a maximum value of 26.7 Mg ha⁻¹, which was associated with a modelled ET value of 1260 mm. The majority of the G×E×M scenarios had GY values ranging between 7.0 and 17.0 Mg ha⁻¹. The modelled ET ranged from 40 mm to 1475 mm. The majority of the G×E×M scenarios had ET values between 200 mm and 700 mm. There was a large number G×E×M scenarios, spanning a wide range ET levels, which resulted in modelled GY outcomes of 0 Mg ha⁻¹. These 0 GY G×E×M scenarios were predominantly associated with ET levels ranging from 50 to 500 mm. However, there were also many G×E×M scenarios associated with ET levels from 50 to 500 mm that resulted in positive GY outcomes. While not evident in the heat map, a large number of the G×E×M scenarios that resulted in severe water deficits during the flowering window were particularly prone to 0 or low GY (see histogram within heatmap in FIG. 5b). The 0 and low GY outcomes were most frequent with ET values below 400 mm. However, low GY outcomes were also predicted for G×E×M scenarios with ET levels beyond 500 mm, particularly when the combination of environmental and management conditions resulted in water deficits during the flowering period.

The relationship between ET and GY was further investigated by quantile regression. The 99% quantile regression (Q99) was estimated as a plausible measure of the water driven yield potential front for maize in the US corn belt. The Q99 asymptote yield value was estimated as 21.47 Mg ha⁻¹ (Table 2). Therefore, the Q99 estimated for the GY-ET framework predicts that for US corn belt environments with sufficient water availability that can achieve high levels of ET and remove other abiotic and biotic constraints 21.47 Mg ha⁻¹ is the 99% repeatable yield potential for maize. For a small number of individual G×E×M scenarios that resulted in an ET of greater than 800 mm a GY greater than 21.47 Mg ha⁻¹ was predicted. However, for the majority of G×E×M scenarios that resulted in an ET greater than 800 m a GY lower than 21.47 Mg ha⁻¹ was predicted. The asymptote yield value for the 80% quantile regression (Q80) was 18.28 Mg ha⁻¹.

TABLE 2
Estimated quantile (80 or 99 percentile) regression parameters (Yp, ET0, TE)
and their standard errors (SE) based on a negative exponential relationship
between grain yield (GY) and evapotranspiration (ET) for the simulation of
GxExM scenarios for the US corn-belt Target Population of Environments (TPE)

| Data Set | Percentile | Yp | SE_{Yp} | ET0 | SE_{ET0} | TE | SE_{TE} |
|---|---|---|---|---|---|---|---|
| GxExM_TPE | 80 | 18.283 | 1.94e⁻³ | 80.54 | 0.04 | 0.00358 | 1.22 × 10⁻⁶ |
| GxExM_TPE | 99 | 21.471 | 5.17e⁻³ | 85.22 | 0.19 | 0.00349 | 1.63 × 10⁻⁶ |

### Example 2

### Genetic Gain: Characterizing G×E×M Interactions for Grain Yield

For the objectives of this study the results of a maize yield ERA study were analysed using the framework of yield front and gap analysis to provide an interpretation of genetic gain for yield in terms of any changes in the yield potential front and the yield gap between potential and realized yield due to drought stress

A hybrid maize ERA experiment was conducted from 3-4 years at three locations; Viluco, Chile, Woodland, Calif., USA and Johnston, Iowa, USA. The three locations were research stations and provided access to information on soil depth and water holding capacity, agronomic management and weather conditions (rainfall, temperature, radiation) required to run a crop growth model suitable to analyse yield potential fronts and yield gaps for the ERA hybrids. At the Viluco and Woodland locations in each year different combinations of plant population and irrigation management were applied to generate a range of environments that differed in level and timing of water availability (Table 3). At the Johnston location different levels of plant population were applied to generate a range of environments (Table 3). A total of 35 environments were generated across the locations and years. For all 35 environments nitrogen fertilizer was applied at levels to avoid nitrogen becoming a significant limiting factor. Thus, all yield potential front and yield gap analyses were conducted assuming that water availability, ranging from severe drought to favourable, was the major environmental variable contributing to the observed variation for grain yield. Timing of water deficit was assessed by estimating the daily S/D ratio, and the total water use estimated by the sum of daily crop ET from planting to physiological maturity, were both calculated using the crop model as described before.

Within each environment a set of ERA maize hybrids was tested for grain yield. The hybrids were all successful Pioneer hybrids with a year of first commercial release spanning the decades from the 1930s through to the 2010s. Within each of the 35 environments the hybrids were evaluated in two replicates of two-row plots. Grain yield was measured using a small-plot combine harvester. To measure grain yield the complete two-row plot was harvested and the shelled grain was weighed and grain moisture determined and yield was calculated from the bulk plot weight and grain moisture and reported as tonnes per hectare at 15.5% grain moisture.

Grain yield data from individual environments and across environments were analysed as a linear mixed model using the ASREML V4.1 software. Within environment spatial analyses were conducted for each environment and across environment analyses were conducted following the multiplicative mixed model methodology. Within the sequence of mixed models applied the hybrids were defined as random terms and Best Linear Unbiased Predictors (BLUPs) were computed for hybrid grain yield across the 35 environments, for hybrid yield in individual environments and for hybrid yield across any subsets of the total set of environments.

Genetic gain for hybrid yield was estimated from the slope of the linear fit of a model factor relating hybrid yield to the year of first commercialisation of the hybrid. Therefore, the classical plant breeding estimate of genetic gain for yield is reported as tonnes (Mega-grams)/hectare/year. To facilitate further analyses of genetic gain the sequence of ERA hybrids were clustered into hybrid groups based on the grain yield results obtained from the ERA study. The grouping was obtained through the analyses of the time series of yield BLUPs for each hybrid across environments using classification and regression trees. The method enabled the identification of discontinuities in the time series, where year provided the information to define a split in a node and to create hybrid groups. The analyses were conducted in R using the package rpart, with year as independent variable and yield BLUPs as the dependent variable. A yield front analysis based on yield across the 35 environments was conducted for each of the hybrid groups to determine whether the yield front had changed with the time and hybrid performance sequence represented by the ERA hybrid groups.

TABLE 3
Description of environments identifying the experiment, location,
year of planting, categorisation of the environments into one
of nine location-year (LY) combinations, plant population defined
in terms of planting density, irrigation defined in terms of the
targeted water regime (WW = Well watered to avoid major water
deficit at flowering time and during the majority of grain filling,
FS = Flowering Stress where irrigation was managed to impose
a severe water deficit predominantly during the flowering window,
GFS = Grain Filling Stress where irrigation was managed to
impose a severe water deficit coincident predominantly with the
grain filling period. For all environments the total water input
during the course of the experiments is defined as the combination
of water supplied by irrigation and rainfall.

| | | | | | Density | | | Rain- |
|---|---|---|---|---|---|---|---|---|
| | | | | | (plants | Irrigation | | fall |
| Env. | Exp. | Location | Year | LY | m⁻²) | Treatment | mm | mm |
| 1 | ERA | Viluco | 1 | 1 | 5.11 | WW | 624 | 11 |
| 2 | ERA | Viluco | 1 | 1 | 8.69 | WW | 624 | 11 |
| 3 | ERA | Viluco | 1 | 1 | 5.11 | FS | 364 | 11 |
| 4 | ERA | Viluco | 1 | 1 | 8.69 | FS | 364 | 11 |
| 5 | ERA | Viluco | 1 | 1 | 5.11 | GFS | 409 | 11 |
| 6 | ERA | Viluco | 1 | 1 | 8.69 | GFS | 409 | 11 |
| 7 | ERA | Viluco | 2 | 2 | 5.11 | WW | 757 | 4 |
| 8 | ERA | Viluco | 2 | 2 | 9.70 | WW | 757 | 4 |
| 9 | ERA | Viluco | 2 | 2 | 5.11 | FS | 499 | 4 |
| 10 | ERA | Viluco | 2 | 2 | 9.70 | FS | 499 | 4 |
| 11 | ERA | Viluco | 2 | 2 | 5.11 | GFS | 390 | 4 |
| 12 | ERA | Viluco | 2 | 2 | 9.70 | GFS | 390 | 4 |
| 13 | ERA | Viluco | 3 | 3 | 9.70 | WW | 659 | 8 |
| 14 | ERA | Viluco | 3 | 3 | 9.70 | FS | 468 | 3 |
| 15 | ERA | Johnston | 3 | 4 | 3.05 | WW | 0 | 310 |
| 16 | ERA | Johnston | 3 | 4 | 5.42 | WW | 0 | 310 |
| 17 | ERA | Johnston | 3 | 4 | 8.12 | WW | 0 | 310 |
| 18 | ERA | Woodland | 3 | 5 | 8.93 | WW | 686 | 4 |
| 19 | ERA | Woodland | 3 | 5 | 2.87 | WW | 686 | 4 |
| 20 | ERA | Woodland | 3 | 5 | 5.10 | WW | 686 | 4 |
| 21 | ERA | Woodland | 3 | 5 | 8.93 | FS | 231 | 4 |
| 22 | ERA | Woodland | 3 | 5 | 2.87 | FS | 231 | 4 |
| 23 | ERA | Woodland | 3 | 5 | 5.10 | FS | 231 | 4 |
| 24 | ERA | Woodland | 3 | 5 | 8.93 | GFS | 148 | 4 |
| 25 | ERA | Woodland | 3 | 5 | 2.87 | GFS | 148 | 4 |
| 26 | ERA | Woodland | 3 | 5 | 5.10 | GFS | 148 | 4 |
| 27 | ERA | Woodland | 3 | 5 | 8.93 | WW | 686 | 4 |
| 28 | ERA | Woodland | 4 | 6 | 8.93 | WW | 510 | 2 |
| 29 | ERA | Woodland | 4 | 6 | 8.93 | FS | 178 | 2 |
| 30 | ERA | Viluco | 4 | 7 | 9.70 | WW | 704 | 1 |
| 31 | ERA | Viluco | 4 | 7 | 5.11 | WW | 704 | 1 |
| 32 | ERA | Viluco | 4 | 7 | 9.70 | FS | 603 | 1 |
| 33 | ERA | Viluco | 4 | 7 | 5.11 | FS | 603 | 1 |
| 34 | ERA | Viluco | 4 | 7 | 9.70 | GFS | 509 | 1 |
| 35 | ERA | Viluco | 4 | 7 | 5.11 | GFS | 509 | 1 |

The grain yield BLUPs for each hybrid in each environment together with the estimated total ET for each environment were used to conduct a yield front analysis. Estimates of the grain yield front for groups of hybrids were obtained by fitting quantile regressions to plots of hybrid grain yield BLUPs against environment mean ET across the 35 environments of the ERA study. Following exploratory comparisons between alternative functions for the quantile regression analyses of the yield-ET data sets the same nonlinear truncated negative exponential function and same R procedure as in the TPE data set were used for the quantile regression analysis a negative exponential function was selected to fit the yield fronts to the sequence of ERA hybrid groups. Following comparisons of different target quantiles, ranging from 80% to 95%, the coefficients for the truncated negative exponential function were estimated at the 80% quantile separately for the ERA hybrid groups.

Results show that for the set of experiments the total ET ranged from a low value of 294 mm for E25 to a high value of 865 mm for E8. The grain yield BLUPs of the maize hybrids across the 35 environments were associated with year of hybrid commercialisation (FIG. 8a). The slope of the linear regression of the hybrid grain yield BLUPs against year of commercialisation provided an estimate of genetic gain of 0.066 Mg ha⁻¹ per year, which is comparable with previous estimates based on earlier studies sampling different plant populations, locations and years in the US corn-belt.

The grouping of the hybrids based on their grain yield performance was associated with the year of commercialisation of the hybrids (FIG. 8a). The only open pollinated variety (OPV) included in the study was identified as a low yielding single member group (G1_OPV). This was followed by a large group of predominantly double cross hybrids (G2_DX). Two groups of older single cross hybrids, commercialized in the decades prior to the incorporation of herbicide and insect protect transgenic traits, were identified (G3_SX, G4_SX). Two groups comprising of more recent single cross hybrids that had different combinations of herbicide and insect protection traits were identified (G5_SXT, G6_SXT). The most recent group (G6_SXT) also contained a number of the AQUAmax hybrids that were developed to have both superior yield under water-limited conditions and high yield potential, and hybrids with higher nitrogen use efficiency. The grouping of the hybrids into the six groups in combination with the estimates of ET for each environment were used as a basis to further investigate the observed genetic gain.

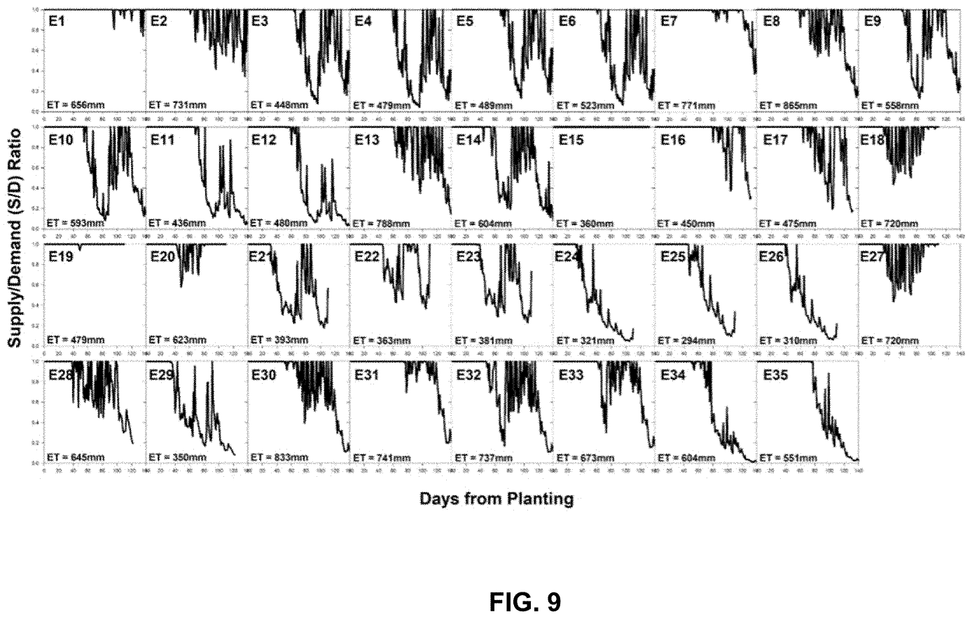

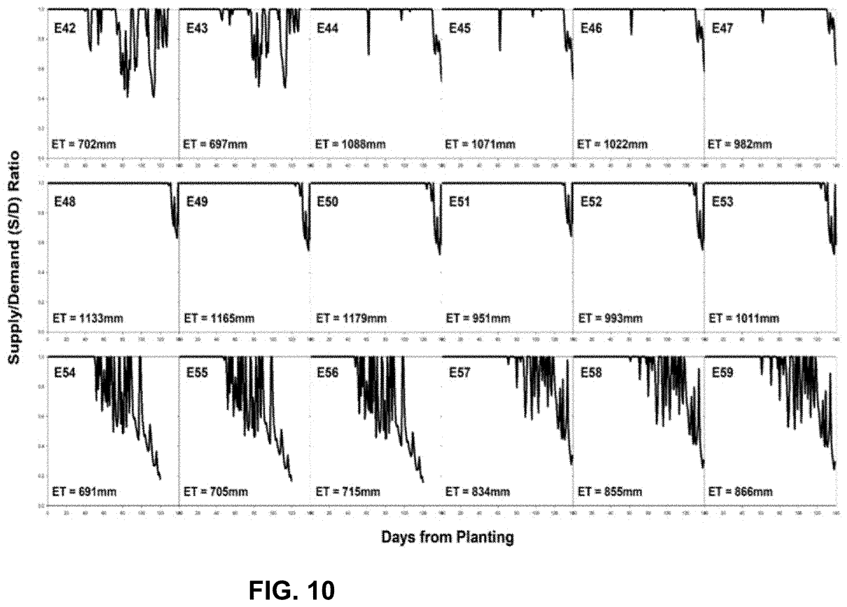

The 35 environments created from the different combinations of plant population, irrigation quantity and timing, location and year sampled a diverse range of water availability regimes that differed in total ET and timing of water deficit as measured by the modelled S/D ratio (FIG. 9). Scatter diagrams comparing hybrid grain yield with environment total ET across the 35 environments were created separately for each of the six hybrid groups (FIG. 8b). For all six groups there was the potential for increased grain yield with increasing ET across the 35 environments. However, the timing of water deficit in relation to flowering time also impacted grain yield, contributing to a range of yield levels observed among hybrids and environments with similar total ET. The influence of timing and intensity of water deficit at flowering is considered in more detail below. The 80% quantile regression (Q80) based on the negative exponential function (equation 1) was estimated separately for each of the six hybrid groups (Table 4, FIG. 8b). The relative shape of the Q80 GY-ET fronts together with the estimates of the three parameters of the negative exponential function for the six hybrid groups provided a basis for reinterpreting the genetic gain for grain yield. The Q80 GY-ET front progressively moved towards increased GY relative to ET from the older to newer hybrid groups (FIG. 8b,c). Therefore, the genetic gain for GY (FIG. 8a) can be investigated in terms of improvements in the GY-ET front (FIG. 8b) and the estimates of the three parameters of the negative exponential function (Table 4). There was no evidence that the ET₀ intercept parameter, representing the minimum level of ET required to obtain yield for the sample of 35 environments, differed among the six hybrid groups. Therefore, the ET₀ parameter was fixed to a common value for the six hybrid groups (Table 4). When the TE parameter was fixed to a common value there was evidence that the Yp asymptote parameter, representing the maximum yield that was achievable with increasing ET, differed among the six hybrid groups (Table 4). When the Yp parameter was fixed to the estimated value by group the differences in the TE parameter were small among the six hybrid groups (Table 4). Direct comparison of the 80% quantile regression yield fronts based on all three parameters estimated for the six hybrid groups revealed a progression in the yield front that was associated with the progression from the older to the newer hybrid groups (FIG. 8b). Superimposing the Q80 GY-ET fronts for the six ERA hybrid groups onto the GY-ET heat map (FIG. 8c) provided a basis for further interpretation of genetic gain for GY.

The empirical GY-ET fronts for all six ERA hybrid groups resided within the distributions of GY and ET values for the G×E×M heat map. The Q80 asymptote yield value for the six hybrid groups progressed from the low value of 9.08 Mg ha⁻¹ for the G1_OPV group to the high value of 18.40 Mg ha⁻¹ obtained for the yield potential asymptote of the G6_SXT hybrid group, which was comparable to the Q80 GY potential asymptote of 18.28 Mg ha⁻¹ for the complete set of G×E×M scenarios (Table 4). The GY-ET front for the complete set of G×E×M scenarios differed from the empirical GY-ET fronts of the six hybrid groups in terms of the ET₀ intercept. The ET₀ intercept for the empirical GY-ET fronts of the six hybrid groups was estimated to be 144.6 mm higher than that obtained for the G×E×M scenarios (Table 4). This result indicates that there is a considerable range of drought (low ET, high stress) Environment-Management scenarios that are predicted to occur with high frequency in the TPE of the US corn belt that were not sampled in the range of Environment-Management scenarios included in the empirical evaluation of the ERA hybrids. Further evaluation of the ERA hybrid sequence in experiments specifically targeted at the low ET drought environments is warranted.

Results from these study can enable defining research strategy and development. Genetic gain was highest for ET greater than 500 mm. Current yield potentials as estimated for the ERA hybrids (Table 4) suggests there is potential to continue improving yields at these levels of ET. These data can clearly inform the decision to invest in breeding for maize in these geographies. In contrast, genetic gain was marginal at best for maize for say ET less than 250 mm. Using methods such as Lean Startup these data can motivate a study to evaluate competing strategies to breed for maize or alternative crops at these levels of ET. Genotype-by-management solutions are clearly a strategy for intermediate ET levels.

TABLE 4
Estimated quantile regression parameters (Yp, ET0, TE) and their
standard errors (SE) based on a negative exponential relationship
between grain yield (GY) and evapotranspiration (ET) for the
80 percentile quantile regression parameters for the six hybrid
groups identified for the ERA hybrid study.

| Data | Percen- | | | | | | |
|---|---|---|---|---|---|---|---|
| Set | tile | Yp | SE_{Yp} | ET0 | SE_{ET0} | TE | SE_{TE} |
| G1_OPV | 80 | 9.081 | 1.151 | 225.1 | 6.58 | 0.00359 | 9.50 × |
| | | | | | | | 10⁻⁴ |
| G2_DX | 80 | 11.916 | 0.206 | 225.1 | 6.58 | 0.00450 | 2.13 × |
| | | | | | | | 10⁻⁴ |
| G3_SX | 80 | 13.668 | 0.182 | 225.1 | 6.58 | 0.00476 | 1.72 × |
| | | | | | | | 10⁻⁴ |
| G4_SX | 80 | 15.901 | 0.448 | 225.1 | 6.58 | 0.00474 | 2.31 × |
| | | | | | | | 10⁻⁴ |
| G5_SXT | 80 | 16.905 | 0.186 | 225.1 | 6.58 | 0.00482 | 1.05 × |
| | | | | | | | 10⁻⁴ |
| G6_SXT | 80 | 18.398 | 0.340 | 225.1 | 6.58 | 0.00483 | 1.66 × |
| | | | | | | | 10⁻⁴ |

### Example 3

### Yield Potential Evaluation

A series of high input experiments was conducted to estimate the yield potential of a set of modern hybrids at high ET levels. The years of commercialisation of each for the experimental hybrids were aligned with the commercialisation period associated with the most recent Group_6 hybrids (see Example 2). A yield potential experiments were conducted from 2016 to 2018 at 3 locations; Viluco, Woodland and Macomb, Ill., USA (Table 5). A range of plant populations was examined at each location. A total of 18 yield potential environments was sampled based on the combinations of location, year and plant population. At Viluco and Woodland drip tape was used to supply water to each row of the experimental plots. At Macomb overhead sprinkler irrigation was used to supply water o the experimental plots. As for the environments of the ERA experiment the CGM was used to estimate any daily incidences of water deficit in terms of the S/D ratio and total ET for each of the 18 environments. After physiological maturity a small plot combine was used to harvest the plots. The shelled grain was weighted, and grain moisture determined, and yield was calculated from the bulk plot weight and grain moisture and reported as tonnes per hectare at 15.5% grain moisture. The yield data were analysed as a linear mixed model using the ASREML V4.1 software. Within environment spatial analyses were conducted for each environment and across environment analyses were conducted following the multiplicative mixed model methodology.

TABLE 5
Description of environments identifying the experiment, location, year of planting,
categorisation of the environments into one of nine location-year (LY) combinations,
plant population defined in terms of planting density, irrigation defined in terms
of the targeted water regime (WW = Well watered to avoid major water deficit
at flowering time and during the majority of grain filling, WW_D =
Well watered with double depths of drip tape (5 cm and 30 cm) with water applications
alternated between the two depths. For all environments the total water input
during the course of the experiments is defined as the combination of water
supplied by irrigation and rainfall.

| | | | | | Density | | | Rainfall |
|---|---|---|---|---|---|---|---|---|
| Env. | Exp. | Location | Year | LY | (plants m⁻²) | Irrigation | (mm) | (mm) |
| 42 | Potential | Macomb | 3 | 8 | 12.34 | WW | 102 | 456 |
| 43 | Potential | Macomb | 3 | 8 | 8.88 | WW | 102 | 456 |
| 44 | Potential | Viluco | 1 | 2 | 13.11 | WW | 1606 | 37 |
| 45 | Potential | Viluco | 1 | 2 | 11.99 | WW | 1606 | 37 |
| 46 | Potential | Viluco | 1 | 2 | 9.66 | WW | 1606 | 37 |
| 47 | Potential | Viluco | 1 | 2 | 8.33 | WW | 1606 | 37 |
| 48 | Potential | Viluco | 2 | 3 | 9.70 | WW | 1334 | 8 |
| 49 | Potential | Viluco | 2 | 3 | 11.83 | WW | 1334 | 8 |
| 50 | Potential | Viluco | 2 | 3 | 13.02 | WW | 1334 | 8 |
| 51 | Potential | Viluco | 2 | 3 | 9.70 | WW_D | 1334 | 8 |
| 52 | Potential | Viluco | 2 | 3 | 11.83 | WW_D | 1334 | 8 |
| 53 | Potential | Viluco | 2 | 3 | 13.02 | WW_D | 1334 | 8 |
| 54 | Potential | Woodland | 2 | 5 | 8.99 | WW | 514 | 5 |
| 55 | Potential | Woodland | 2 | 5 | 10.65 | WW | 514 | 5 |
| 56 | Potential | Woodland | 2 | 5 | 11.83 | WW | 514 | 5 |
| 57 | Potential | Woodland | 3 | 6 | 8.99 | WW | 686 | 70 |
| 58 | Potential | Woodland | 3 | 6 | 10.65 | WW | 686 | 70 |
| 59 | Potential | Woodland | 3 | 6 | 11.83 | WW | 686 | 70 |

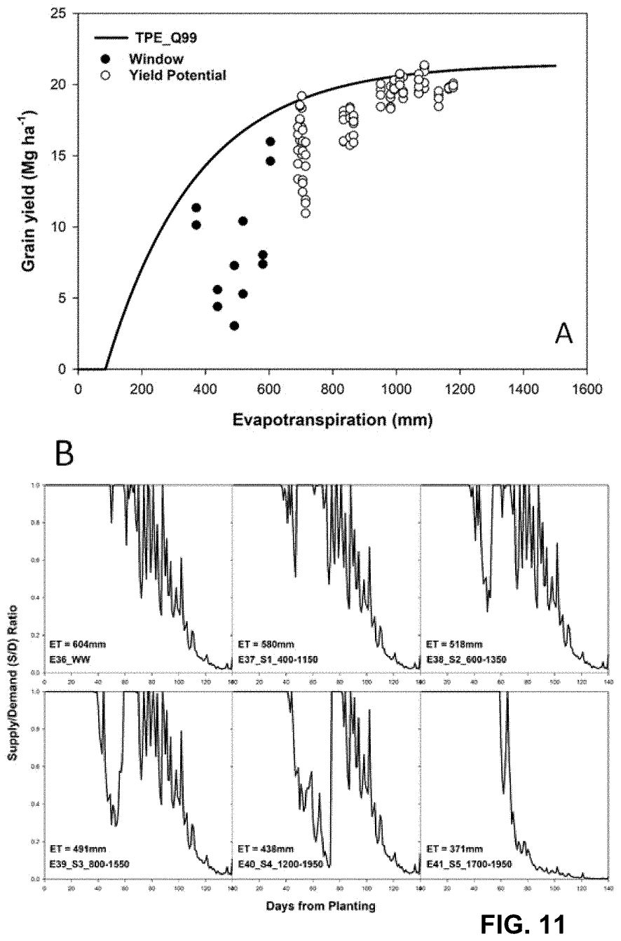

The highest yield potential estimates predicted from the full set of modelled G×E×M scenarios (FIG. 5b) occurred at ET levels beyond those that were sampled in the 35 ERA experiment environments (FIG. 8c). The yield potential experiment was conducted to provide an empirical test of the GY predictions at high ET levels. From Year 1 to Year 3, a series of experiments was conducted at three locations (Table 5; environments 42 to 59) to generate a set of high ET environments to evaluate the yield potential of modern elite maize hybrids. The ET ranged from 691 mm (E54) to 1179 mm (E50) (FIG. 11). While irrigation was supplied to reduce the incidence of water deficits the modelled, S/D ratios indicated that for a number of the environments irrigation supply was insufficient to meet the demand of the crop canopy, particularly towards the end of the season. While transient water deficits were indicated by the S/D ratios, the empirical GY estimates obtained from the yield potential experiments indicated that the highest experimental GY values obtained from each experiment, given the ET level, were comparable to the predicted yield potential of the environments based on the Q99 GY-ET front obtained for the full set of G×E×M scenarios (FIG. 11a).

The results from this study illustrates that even at 700 mm of water availability the wrong choice of hybrid and management can lead to performance well below that it could attainable from the available environmental resources. From a breeding-agronomy perspective, these results suggest that there are opportunities to identify genotype-management technologies that can lead to technologies that fully utilize the environmental resources delivering value to farmers.

### Example 4

### Yield Under Drought Stress when Vary with Development Stage: Genotype-by-Timing of Irrigation Interaction

An experiment based on a series of managed six managed water experiments was conducted at Viluco in Year 2 to estimate the impact of different timing of water deficit during development on the yield of a drought tolerant (P1151—hybrid 1) and a drought sensitive (P1197—hybrid 2) hybrid (Table 6). A sequence of five water deficit environments were designed to follow an irrigation water management protocol. The objective of the different irrigation strategies was to create a sequence of water deficit environments that differed in the timing of an imposed water deficit window in relation to the reproductive development and flowering window of the two hybrids. The timing and intensity of the water deficit was adjusted by changing the quantity and timing of irrigation. A well-watered control environment was also created. Twenty replicates of each hybrid were grown as two-row plots in each environment. As for the environments of the ERA and yield potential experiments the CGM was used to estimate daily incidences of water deficit in terms of the S/D ratio and total ET for each of the six environments. After physiological maturity a small plot combine was used to harvest the plots. The shelled grain was weighted and grain moisture determined, and yield was calculated from the bulk plot weight and grain moisture and reported as tonnes per hectare at 15.5% grain moisture. The yield data were analysed as a linear mixed model using the ASREML V4.1 software. Within environment spatial analyses were conducted for each environment. Since there were only two hybrids included in the experiment the hybrids were treated as fixed for the mixed model analyses of variance.

TABLE 6
Description of environments identifying the experiment, location, year of planting,
categorisation of the environments as location-year (LY) combinations, plant population
defined in terms of planting density, irrigation defined in terms of the targeted water
regime (WW = Well watered to avoid major water deficit at flowering time and during
the majority of grain filling, WW = Well watered, FS = Flowering Stress where
irrigation was managed to impose a severe water deficit predominantly during the
flowering window, GFS = Grain Filling Stress where irrigation was managed to impose a
severe water deficit coincident predominantly with the grain filling period (FS_S1,
FS_S2, FS_S3, FS_S4, GFS_S5 identified as a sequence of five stress treatments
(S1 to S5) where the timing of the major water deficit was imposed by withholding
irrigation water as a moving time window). For all environments the total water input
during the experiments is defined as the combination of water supplied by irrigation and
rainfall.

| | | | | | Density | | | Rainfall |
|---|---|---|---|---|---|---|---|---|
| Env. | Experiment | Location | Year | LY | (plants m⁻²) | Irrigation | (mm) | (mm) |
| 36 | Window | Viluco | 2017 | 3 | 9.70 | WW | 588 | 3 |
| 37 | Window | Viluco | 2017 | 3 | 9.70 | FS_S1 | 453 | 3 |
| 38 | Window | Viluco | 2017 | 3 | 9.70 | FS_S2 | 432 | 3 |
| 39 | Window | Viluco | 2017 | 3 | 9.70 | FS_S3 | 435 | 3 |
| 40 | Window | Viluco | 2017 | 3 | 9.70 | FS_S4 | 454 | 3 |
| 41 | Window | Viluco | 2017 | 3 | 9.70 | GFS_S5 | 391 | 3 |

The GY-ET results of the modelled G×E×M scenarios (FIG. 4) indicated that the coincidence of a water deficit during the flowering period of the maize hybrids can result in a reduction in realised yield relative to the potential yield for a given ET level. The flowering window experiment was conducted to provide an empirical test of the GY impact of water deficits coincident with the flowering window (Table 5; environments 36 to 41). For the six environments the ET ranged from 371 mm (E41) to 604 mm (E36; the control) (FIG. 11b). The S/D ratios for the six environments indicated a water deficit that resulted in a S/D ratio <1.0 coincident with the flowering window of the hybrids for E37 to E40. For both E36 and E41 the S/D ratio decreased below 1.0 after the flowering window. The empirical GY estimates obtained from the flowering window experiment environments were compared to the predicted GY, given the ET level, based on the Q99 GY-ET front obtained from the full set of G×E×M scenarios (Table 2; FIG. 11a). For both environments where irrigation was supplied to minimise water deficits coincident with flowering the empirical GY was slightly lower than the predicted GY based on the Q99 GY-ET front. However, for the four environments where irrigation was managed to impose a water deficit coincident with flowering the empirical GY was greatly reduced relative to the predicted GY based on the Q99 GY-ET front (FIG. 11a, full dots).

At Viluco, environment-location LY-3 (Table 3, 5, 7) the two hybrids were tested in 14 different management combinations. Based on the combination of the daily S/D ratio and the grain yield levels achieved by the two hybrids relative to the attainable yield prediction based on the modelled ET level and Q80 quantile regression a yield reduction was inferred for seven (E41, E40, E39, E38, E37, E14, E36) of the management treatments and for the other seven (E13, E51, E52, E53, E48, E49, E50) a yield level above the Q80 predicted attainable yield was inferred. Thus, a yield gap was inferred for the seven environments with observed yield below the Q80 predicted yield for at least one of the hybrids. For the seven environments where the observed yield was above the Q80 predicted yield there was no consistent grain yield advantage for either hybrid. However, for the seven environments where the observed yield was below the Q80 predicted yield P1151 resulted in a higher grain yield than P1197 (FIG. 7b). Thus, the yield gap could be reduced by hybrid choice in these water limited environments. The yield gap could also be reduced in these environments by adjusting irrigation strategies to minimise the coincidence of water deficits with the flowering window. This is further emphasised by the grain yield results obtained in E41, where less irrigation was applied than in the four management treatments E40, E39, E38 and E37, while higher grain yield was achieved for both hybrids through avoiding severe water deficits during the flowering window. Thus, through combinations of hybrid choice, management strategy and hybrid-management combination choice at LY-3 there were a number of opportunities to reduce the yield gap between realised grain yield and the achievable and potential grain yield for the crop available water. Furthermore, it should be possible to anticipate hybrid susceptibilities to water deficit through simulation and make better selections for genotype-management technologies.

TABLE 7
Variance components (± standard errors) for simulated grain yield (GY)
and evapotranspiration (ET) for two selected grids; Grid 10018 selected based on
large Genotype (G) source of variance for GY relative to Genotype by
Management (GxM) source of variance, Grid 7453 selected based on large
Genotype by Management (GxM) source of variance for GY relative to Genotype
(G) source of variance.

| | Grid | | | |
|---|---|---|---|---|
| | 10018 | | 7453 | |
| | Grain Yield | Evapotranspiration | Grain Yield | Evapotranspiration |
| Source | (Mg ha⁻¹) | (mm) | (Mg ha⁻¹) | (mm) |
| Year (Y) | 0.75 ± 0.16 | 884.9 ± 180.5 | 1.51 ± 0.32 | 1176.5 ± 250.5 |
| Management (M) | 0.95 ± 0.41 | 2980.2 ± 1272.6 | 4.05 ± 1.74 | 12928.9 ± 5523.6 |
| YxM | 0.24 ± 0.01 | 204.7 ± 12.4 | 0.91 ± 0.05 | 904.7 ± 54.6 |
| Genotype (G) | 3.22 ± 0.21 | 6555.2 ± 421.0 | 0.50 ± 0.04 | 4870.7 ± 331.5 |
| GxM | 0.07 ± 0.001 | 106.2 ± 2.1 | 2.18 ± 0.04 | 3556.9 ± 68.9 |
| GxY | 0.21 ± 0.002 | 253.4 ± 2.4 | 0.53 ± 0.01 | 241.8 ± 2.5 |
| Residual | 0.23 ± 0.001 | 184.8 ± 0.5 | 0.71 ± 0.002 | 403.7 ± 1.1 |

### Example 5

### Gap Analyses Applied to Large Simulated Datasets: Identifying Genotype-by-Management Opportunities to Attain Target Production Efficiencies

Two approaches for yield productivity gap analysis include: (1) empirical data, and (2) simulated data. An extension of previous gap analysis applications that is considered here is a focus on characterising the potential and relative opportunities to reduce yield productivity gaps by G, M and GxM individually and in combination.

The combination of the experimental results obtained from the ERA, Yield Potential and Window experiments together with Q80 and Q99 quantile regression predictions for the G×M×E scenarios were used to demonstrate the application of the gap analysis methodology (see examples above). By comparison of the experimental grain yield results with the predicted grain yield, based on the Q80 and Q99 quantile regressions for the modelled ET, each environment could be classified as either meeting (grain yield between the Q80 and Q99 prediction) or not meeting the expectation (grain yield below the Q80 prediction) given the modelled ET level for each of the 59 environments (Table 3, 5, 6). Those environments not meeting the expectation then become the environments of focus for identification of G-M strategies for closing the yield gap.

The grain yield and ET results obtained from the simulation of maize G×E×M for the 2265 30 km by 30 km grids were also used to undertake a gap analysis applied to data generated using simulation for each of the 2265 grids. The investigation of the simulated yield results for the G×E×M scenarios. Results can provide a referencing framework to: (1) assist interpretation of any empirical G×E×M analyses conducted at the same scale, and (2) to evaluate the relative merits of alternative Genotype, Management and Genotype-Management technology options to achieve target levels of on-farm crop productivity. Here examples, selected from the full set of 2265 grid results, are used to demonstrate the potential of the approach to quantify and identify the opportunities to exploit G, M and GxM variation to reduce yield productivity gaps at the scale of a grid.

The first step after simulation (FIG. 6) was analysis of sources of variance for each grid and summarisation of the results for the full set of 2265 grids. At the grid level a mixed model analysis of the simulated grain yield and ET data was conducted applying the model (with all terms except for mu treated as random):

Tᵢⱼₖ=mu+Gᵢ+Mⱼ+Yₖ+(GM)ᵢⱼ+(GY)ᵢₖ+(MY)ⱼₖ+eᵢⱼₖ

where Tᵢⱼₖ is the Trait (Grain yield or ET) value for genotype i in management j in year k, mu is the fixed effect for the overall mean, Gᵢ is the main-effect for genotype i, assumed to be N(0,σ2G), Mⱼ is the main-effect for management j, assumed to be N(0, σ2M), Yₖ is the main-effect for year k, assumed to be N(0, σ2Y), (GM)ᵢⱼ is the Genotype-by-Management interaction effect for Genotype I and Managementj, assumed to be N(0, σ2GM), (GY)ᵢₖ is the genotype-by-year interaction effect for Genotype I and Year k, assumed to be N(0, σ2GY), (MY)ⱼₖ is the Management-by-Year interaction effect for Management j and Year k, assumed to be N(0, σ2MY), and eᵢⱼₖ is the residual effect for Genotype I in Management j and Year k, assumed to be N(0,σ2e).

The estimates of the variance components for all 2265 grids were used to construct boxplots to visualise the distributions of the variance components for grain yield and ET across all 2265 grids (FIG. 6a). Also, for each selected grid the simulated GY was plotted against the simulated ET for all G×M×E scenarios to generate a GY-ET heat map at the grid level. BLUPs were computed for GY and ET for genotype main-effects (G), management main-effects (M) and Genotype-Management combinations (GxM) (FIG. 6b). The graphical views created for the chosen grids (FIG. 6b, cases 1 and 2) were then investigated to identify the potential yield productivity benefits that can be predicted for changes in G, M and GxM strategies and the associated predicted impact on ET.

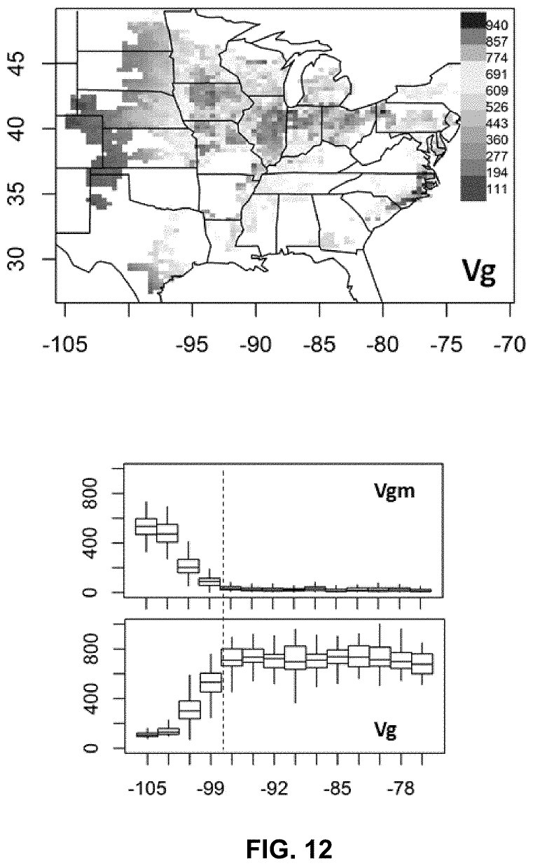

To search for opportunities to increase yield gain vary with location or target environment or geography, the variance components for GY and ET for each of the 2265 grids were explored using boxplots. Variance components provided a summary of the relative sizes and distribution of the sources of variation within the simulated G×E×M data set (FIG. 6a). The genotypic variance component was on average across the region the largest source of variance for both GY and ET. The management variance component was the second largest source of variance for both GY and ET. For GY the management variance component was on average similar in magnitude to the year variance component, whereas the year variance component was smaller for ET. The GxM variance component was on average smaller than both the genotypic and management variance components. However, the extreme values of the boxplots indicated that for some of the grids the GxM variance component could be as large as the genotypic variance component. Further investigation of the variance components based on their ratios for the individual grids indicated that the relative importance of the genotypic, management and GxM sources of variance differed among the 2265 grids and their relative magnitude was strongly associated with longitude with a smaller association indicated for latitude (FIG. 12). This suggests that the relative effectiveness of different strategies for reducing the yield gap, based on the contributions from genotype, management and GxM interactions through their impact on effective crop water use, as quantified by ET, can differ for the grids across the US corn belt.

To further explore the proposition that the effectiveness of strategies to close the yield gap will depend on location across the US corn belt individual grids were identified based on the relative sizes of the genotypic, management and GxM variance components for GY. For each selected grid the GY and ET BLUPs were computed for the 488 genotypes (G_BLUPs), the 12 managements (M_BLUPs) and the 5856 GxM combinations (GxM_BLUPs). Scatter diagrams were constructed to compare GY and ET for the G_BLUPs, M_BLUPs and GxM_BLUPs (FIG. 6).

Case 1 in FIG. 6b was identified based on the large ratio of the genotypic variance component relative to the management component for GY. For this grid the G_BLUPs covered a wider range of levels of ET and had a higher associated range of levels of GY than the M_BLUPs. There was little GxM interaction. Therefore, the relative GY values and ET levels were well predicted by the combination of the G_BLUPs and M_BLUPs. Therefore, in this case there was more capacity to close the yield gap by choosing among the 488 genotypes than by choosing among the 12 management strategies considered.

Case 2 in FIG. 6b was identified based on the large ratio of the GxM variance component relative to the sum of the genotypic and management variance component. For this grid the M_BLUPs covered a wider range of levels of ET and had a higher associated range of levels of GY than the G_BLUPs. Therefore, for case 2, in contrast to case 1, there was more capacity to close the yield gap by choosing among the 12 management strategies than by choosing among the 488 genotypes. For case 1, the strong contribution of GxM interactions for GY and ET required consideration of the GxM_BLUPs to identify the preferred strategy to close the yield gap. The pattern of the GxM_BLUPs for GY and ET suggests that within case 2 where a grower has access to full irrigation capacity choices of plant population and genotype would differ to the choices made by a grower that had access to only a limited irrigation strategy, which in turn would differ to the choices made by a grower with o access to irrigation. Further, this could represent different field choices for individual growers.

### Example 6

### Digital Gap Analysis: Grain Yield

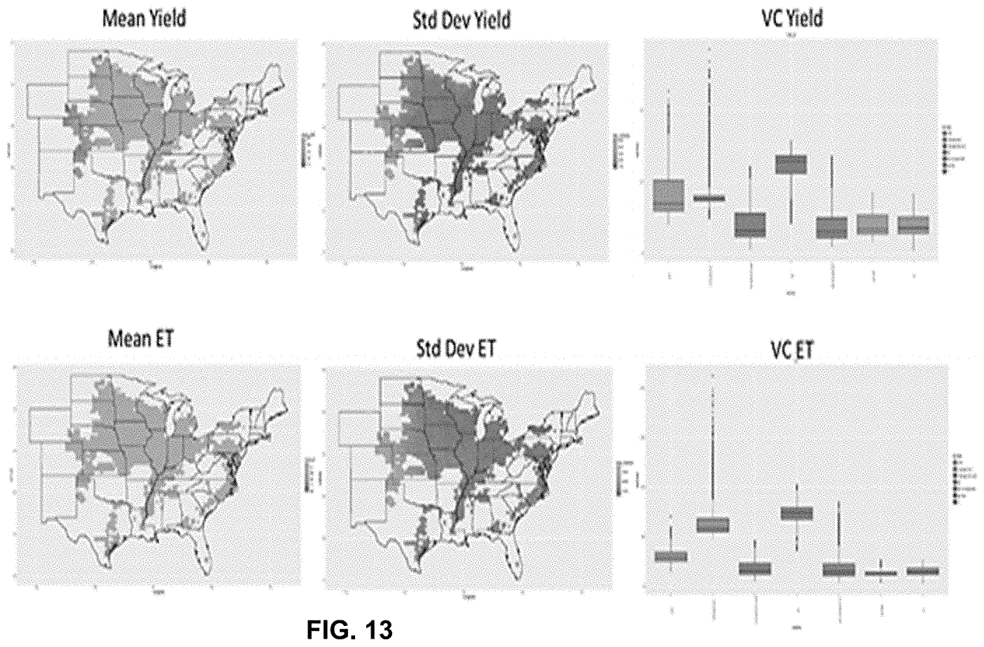

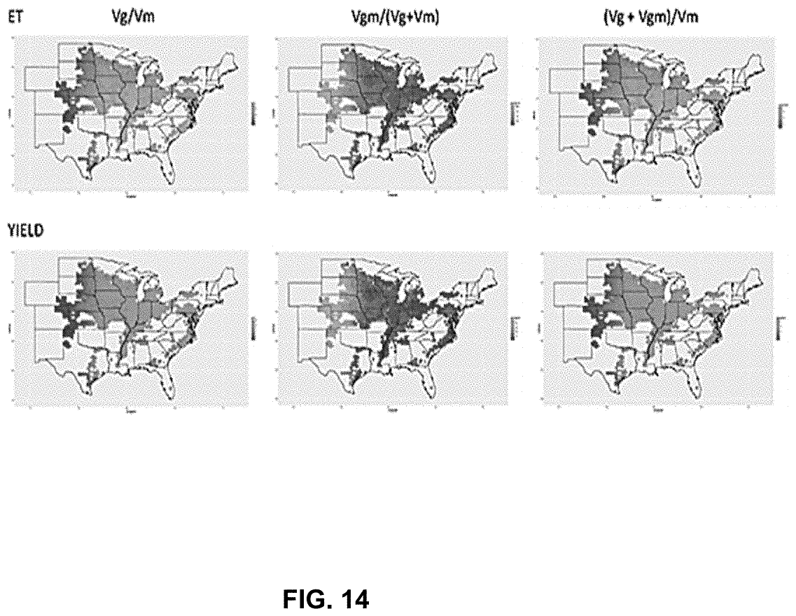

The boxplots for the variance components for GY and ET for each of the 2265 grids provided a summary of the relative sizes and distribution of the sources of variation within the simulated G×E×M data set (FIG. 13). The genotypic variance component was on average the largest source of variance for both GY and ET. The management variance component was the second largest source of variance for both GY and ET. For GY the management variance component was on average similar in magnitude to the year variance component, whereas the year variance component was smaller for ET. The GxM variance component was on average smaller than both the genotypic and management variance components. However, the extreme values of the boxplots indicated that for some of the grids the GxM variance component could be as large as the genotypic variance component. Further investigation of the variance components based on their ratios for the individual grids indicated that the relative importance of the genotypic, management and GxM sources of variance differed among the 2265 grids and their relative magnitude was strongly associated with longitude with a smaller association indicated for latitude (FIG. 14). This indicates that the relative effectiveness of different strategies for reducing the yield gap, based on the contributions from genotype, management and GxM interactions through their impact on effective crop water use, as quantified by ET, can differ for the grids across the US corn belt.

To further explore the proposition that the effectiveness of strategies to close the yield gap will depend on location across the US corn belt individual grids were identified based on the relative sizes of the genotypic, management and GxM variance components for GY. For each selected grid the GY and ET BLUPs were computed for the 488 genotypes (G_BLUPs), the 12 managements (M_BLUPs) and the 5856 GxM combinations (GxM_BLUPs). Scatter diagrams were constructed to compare GY and ET for the G_BLUPs, M_BLUPs and GxM_BLUPs (FIG. 14).

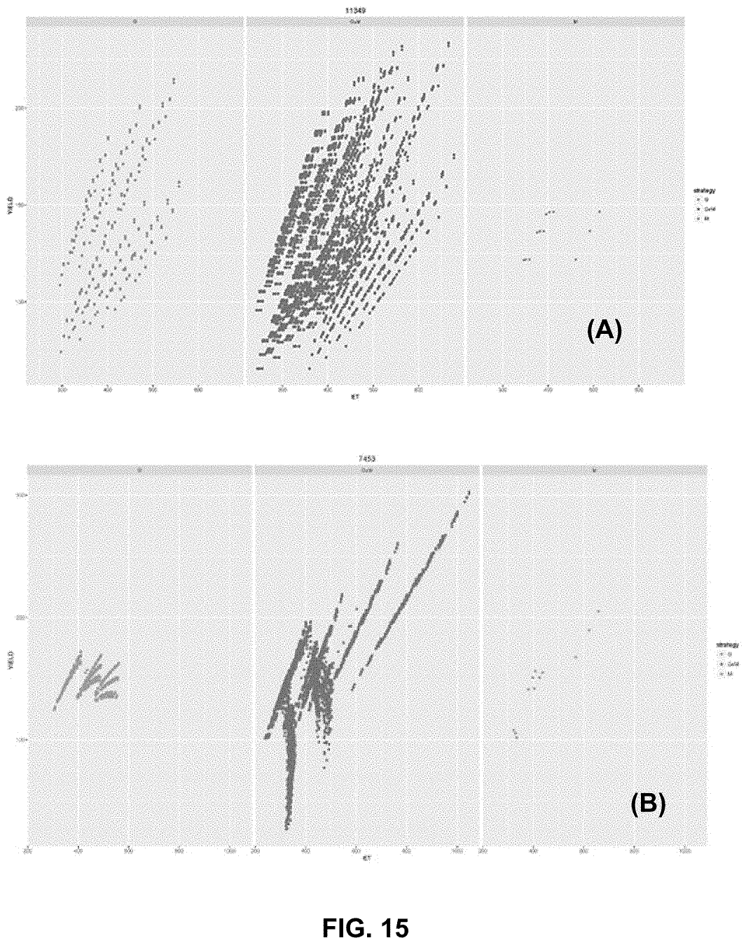

Grid 11349 was identified based on the large ratio of the genotypic variance component relative to the management component for GY (FIG. 15a). For this grid the G_BLUPs covered a wider range of levels of ET and had a higher associated range of levels of GY than the M_BLUPs. There was little GxM interaction. Therefore, the relative GY values and ET levels were well predicted by the combination of the G_BLUPs and M_BLUPs. Therefore, for grid 11349 there was more capacity to close the yield gap by choosing among the 488 genotypes than by choosing among the 12 management strategies considered.

Grid 7453 was identified based on the large ratio of the GxM variance component relative to the sum of the genotypic and management variance component (FIG. 14b). For this grid the M_BLUPs covered a wider range of levels of ET and had a higher associated range of levels of GY than the G_BLUPs. Therefore, for grid 7453, in contrast to grid 11349, there was more capacity to close the yield gap by choosing among the 12 management strategies than by choosing among the 488 genotypes. For grid 7453, the strong contribution of GxM interactions for GY and ET required consideration of the GxM_BLUPs to identify the preferred strategy to close the yield gap. The pattern of the GxM_BLUPs for GY and ET suggests that within grid 7453 where a grower has access to full irrigation capacity choices of plant population and genotype would differ to the choices made by a grower that had access to only a limited irrigation strategy, which in turn would differ to the choices made by a grower with o access to irrigation. Further, this could represent different field choices for individual growers.

### Example 7

### Machine Learning, Deep Learning Based Artificial Intelligence Computing Systems to Synchronize Breeding Parameters with Agronomic Management Practices

In an embodiment, one or more of the variables described herein for example, genotypic information, environmental factors, and/or management practices can be fed into a machine learning or deep learning algorithm. For example, a neural network architecture for computing one or more predicted breeding values from one or more crop related management practice inputs. The neural networks are configured to synthesize or learn from a plurality of inputs to produce an output—for example, one or more inputs to a crop growth model (CGM) can be modeled using machine learning approaches involving Bayesian algorithms. One or more variables in the algorithms can have weights that are applied to each equation and optimized as the neural network is trained. Based on the amount of training information the deep learning models or networds get better at producing more helpful outputs.

Individual machine learning networks (e.g., artificial neural networks—ANN; Convolutional Neural Networks (CNN)s) are described herein at general terms based on inputs, outputs, and type of neural network. Based on the various inputs, such as for example, genetic haplotype information and field effects realized from one or more agronomic management practices, one of ordinary skill in the art given data on the inputs, outputs, and type of machine or deep learning modules would be able to construct working embodiments.

In an embodiment, deep neural network includes a plurality of input factors that may be used to train the synchronized breeding by management practices. These factors include for example, breeding histories, pedigree, QTLs, SNPs, haplotypes, yield, environmental classifications, fertilizer input, water availability, and other agronomic or breeding components.

Irrigation, plant population density, planting date, nutrient application (e.g., N, P, K), other seed applied/soil applied components such as seed treatments, agricultural biologicals, crop rotations, and other practices form the agronomy management practice described herein.

Training data generally refers to datasets that are used to train specific deep learning networks, such as for example, neural network. Each dataset may correspond to set of actual yield values and the underlying management practice components for one or more crops. Yield values for example, represent grain yield. Other values such as biomass, pollen shed, silking can also be utilized. Training datasets can be used with various types of machine learning algorithms such as supervised learning, unsupervised learning, semi-supervised learning, and reinforcement learning. Neural network algorithm is an example of supervised learning—where a special purpose computer or a computing system is provided with training data containing the input/predictors along with the correct output. From the training data the computer/algorithm should be able to learn the patterns. Supervised learning algorithms model associations and dependencies between the target prediction output and the input features such that the output values for new data based on those previous associations that the network learned from. Training datasets can include measured data, simulated data, or a combination thereof.

In an embodiment, training data also includes for example, genetic associations for grain yield and one or more of agronomic parameters such as planting density, nitrogen application, nutrient inputs, water availability and one or more management practice data. Not each of the data types is needed to train the deep learning network. For example, datasets that include crop by yield and soil type data are capable of evaluating the effects on predicted total grain yield.

Datasets may include data obtained from various crop field and/or greenhouse evaluations. These data include for example, geographical location, weather history, historical precipitation, GDU, soil type, soil moisture, soil temperature, management practices, and additional information such as for example, crop rotation, applied nitrogen, cover crop presence or practice and other agronomically relevant parameters. Agricultural special purpose computer system capable of monitoring, measuring and analyzing additional data from a plurality of breeding centers are described herein. For example, such computers may receive one or more of such data either directly from the plurality of breeding centers or evaluation stations or sensors or input by users.

In an illustrated embodiment, CGM simulated G×E×M scenarios and their predicted yield potential front and yield gap distributions are modeled using a neural network algorithm. In another embodiment, results obtained from the simulation of G×E×M for grain yield of maize for the US corn-belt TPE and the comparisons with the experimental results are modeled using a machine or deep learning approach to discuss opportunities for applying an integrated approach across breeding and agronomy to enhance understanding and prediction of G×E×M interactions and the creation and identification of desirable genotype-management combinations that improve maize yield productivity and stability by mitigating the negative effects of drought across the US corn-belt.

1. A method of accelerating synchronized breeding and management practice, the method comprises:
providing an integrated quantitative framework across breeding and agronomy management, wherein the quantitative framework comprises a breeding component and at least two agronomic management components that form a gap analysis;
predicting one or more improvements in crop productivity from the quantitative framework strategies; and
combining a genetic component with the agronomy management components to synchronize breeding such that a breeding plant population is selected based on the gap analysis.

2. The method of claim 1, wherein the quantitative framework comprises selecting a population of plants for breeding based on a predicted performance of one or more of the population of plants under a targeted agronomic management practice.

3. The method of claim 2, wherein the agronomic management practice is selected from the group consisting of nutrient management, water management, population density and crop rotation.

4. A method of synchronized breeding and agronomy for increasing yield, the comprises:
a. proving a crop model or other quantitative simulation data to formulate one or more genotype by management approaches to breeding;
b. selecting a subset of selected agronomic management conditions based on the crop growth model or the quantitative simulation data applicable to one or more genotypes of a population of plants at an early stage in a breeding pipeline;
c. growing one or more members of the population of plants in one or more crop growing environments comprising the agronomic management conditions;
d. applying one or more selection criteria to the population of plants grown in the crop growing environments such that the selected plants are capable of expressing their genetic potential in the selected agronomic management conditions;
e. selecting the plants for further breeding advancement, wherein the selected plants are better suited for a target environment or a target agronomic management practice based on the performance of the plants in the subset of the crop growing environments.

5. A method of integrating one or more agronomic practices (management) into early-stage breeding pipeline, the method comprising non-sequentially applying one or more crop growing environmental (E) and management (M) to a population of plants comprising genotypic variations (G), wherein the crop growing environmental conditions are informed by a crop growth model or a statistically significant quantitative framework, or a simulation or a combination of the foregoing; and selecting a subset of the population of the plants for further breeding advancement.

6. The method of claim 5, wherein the one or more agronomic practices include a practice selected from the group consisting of irrigation, planting date, plant population, plant nutrition, defoliation, harvest, crop sequence, crop rotations, crop combinations in one field, one farm, one geography or multiple fields, farms and geographies, or a combination of the foregoing.

7. The method of claim 5, wherein the environmental conditions include water stress, nitrogen stress, pest pressure, cold stress, heat stress, salinity, moisture, soil type, or a combination thereof.

8. The method of claim 5, wherein the quantitative method includes one or more of methods based on crop growth models, statistical models including machine learning, remote sensing, and any combination suitable to generate a genotype×environment, genotype×management, and genotype×management systems.

9. (canceled)

10. (canceled)

11. (canceled)

12. (canceled)

13. A specialized computing system for integrated breeding parameters and agronomic management practice, the system comprising: a memory; a first deep learning network stored in the memory, configured to compute first agronomy management practice effect on crop yield or genetic gain, the agronomy practice data as input;
a second deep learning network stored in the memory, configured to compute a second management practice effect on crop yield using the second management practice data as input;
a third deep network stored in the memory, configured to compute a third management practice effect on crop yield using the third management practice data as input;
a master deep learning network stored in the memory, configured to compute one or more yield values using the first, second, and third management practices effect on crop yield using the first, second, and third management practice data as inputs;
one or more processors communicatively coupled to the memory, configured to execute one or more instructions to cause performance of: receiving a particular dataset relating to one or more agricultural fields, wherein the particular dataset comprises particular first, second and third management practice data;
using the first deep learning network, computing the first management practice effect on crop yield for the one or more agricultural fields from the first management practice data;
using the second deep learning network, computing the second management practice effect on crop yield for the one or more agricultural fields from the second management practice data;
using the third deep learning network, computing the third management practice effect on crop yield for the one or more agricultural fields from the third management practice data; and
using the master deep learning network, computing one or more predicted yield values for the one or more agricultural fields from the first, second, and third management practice effects on crop yield.

14. The system of claim 13, wherein the first management practice data comprises nitrogen management; wherein the first deep learning network comprises a neural network configured to associations between the first management practice that are correlated to effects on crop yield.

15. The system of claim 13, wherein the crop is maize, soy, canola, cotton, rice, wheat, sorghum, and sunflower.

16. The system of claim 13, wherein the one or more breeding parameters include genotypic and/or phenotypic data.

17. The system of claim 16, wherein the genotypic data includes a genome sequence information selected from the group consisting of SNP, QTL, RNA-seq, short read genomic sequencing, marker data, long read genome sequence information, methylation status, gene expression values, and indels.

18. The system of claim 16, wherein the agronomy management practice component is selected from the group consisting of irrigation, plant population density, planting date, nutrient application, seed or soil applied agricultural biologicals, crop rotations, and targeted in-season crop protection agent.

19. (canceled)

20. (canceled)

21. The system of claim 16, wherein the management practice for crop yield comprises one or more plants in a breeding pipeline, comprises growing the plants in a crop growing environment, wherein the crop growing environment includes one or more agronomic practices tailored to pre-selected agronomic management parameters for improved performance that are targeted to one or more locations, conditions, and or management practices, wherein the agronomic practices are pre-selected based on crop growth model, empirical simulation, statistical modeling, a quantitative model or a combination thereof.

22. The system of claim 21, wherein the plants are at a breeding stage considered as early stage in which the commercial value or potential of the plants is not well established.

23. The system of claim 21, wherein the plants are progeny of early stage inbreds.

24. The system of claim 21, wherein the agronomic practices and the genetic gain selection are performed non-sequentially.
